SEQUENCE LISTING (10 sequences)

SEQ ID NO: 1 - DNA, 22 bp, Artificial Sequence
  primer
aagagagaggccctcagttgct

SEQ ID NO: 2 - DNA, 22 bp, Artificial Sequence
  primer
ttgtgagggagatgctcagtgt

SEQ ID NO: 3 - DNA, 20 bp, Artificial Sequence
  primer
gacaggccctactgggaatg

SEQ ID NO: 4 - DNA, 24 bp, Artificial Sequence
  primer
cgttgtcaagaaacactgaagaca

SEQ ID NO: 5 - DNA, 18 bp, Artificial Sequence
  primer
cggcttcagcgagtgcat

SEQ ID NO: 6 - DNA, 19 bp, Artificial Sequence
  primer
cggtgttaacgccctcaca

SEQ ID NO: 7 - DNA, 18 bp, Artificial Sequence
  primer
ggaggcggtgcagttcct

SEQ ID NO: 8 - DNA, 22 bp, Artificial Sequence
  primer
ggagtggtaaagcagcttcatc

SEQ ID NO: 9 - DNA, 22 bp, Artificial Sequence
  primer
ggacagacgagtgcctcagttc

SEQ ID NO: 10 - DNA, 24 bp, Artificial Sequence
  primer
tcatggcttcaagctcatcctcct

(19) United States
(12) Patent Application Publication
(10) Pub. No.: US 2004/0254152 A1
(43) Pub. Date: Dec. 16, 2004

(54) Prevention of deficits in neurogenesis with anti-inflammatory agents

(76) Inventors: Michelle L. Monje, (US); Theo D. Palmer, (US)

Correspondence Address: BOZICEVIC, FIELD & FRANCIS LLP, 1900 UNIVERSITY AVE, SUITE 200, EAST PALO ALTO, CA, 94303, US

(21) Appl. No.: 10/826,472

(22) Filed: Apr. 16, 2004

Related U.S. Application Data
(60) Provisional application No. 60/463,769, filed on Apr. 17, 2003.
(60) Provisional application No. 60/519,562, filed on Nov. 12, 2003.

(51) Int. Cl. A61N 5/00; A61K 31/60; A61K 31/415

(52) U.S. Cl. 514/165; 514/406; 514/569; 600/1

(57) ABSTRACT

Methods are provided for protecting an individual from adverse long-term effects of neuroinflammation. Inflammatory blockade maintains neurogenesis capability after cranial irradiation by reducing the negative effects of activated microglia on neural precursor cells. These findings have broad implications for a variety of diseases of cognition, involving neuroinflammation and precursor cell dysfunction.

### BACKGROUND

Hippocampal neurogenesis occurs throughout life and the balance of neuronal loss and birth is essential in generating the plasticity necessary for new memory formation. The generation of new neurons within the hippocampus is mediated by proliferating neural stem/progenitor cells that are exquisitely sensitive to local signaling. Stem cells represent the most immature cell necessary for neurogenesis. This cells gives rise to more restricted precursors or progenitor cells and ultimately these progenitors differentiate into new functional neurons. These cells produce neurons in response to signals received from surrounding cells as well as humoral signals from circulating hormones, cytokines, and growth factors. Gross alterations in local microenvironments may allow ectopic neurogenesis to occur, or even block essential neurogenesis, leading to deficits in neurogenesis-dependent functions, such as learning and memory. Within this relatively new field of study, a paradigm of neural stem/progenitor cell dysregulation is emerging. Stress and the accompanying changes in stress hormones orchestrated by the hypothalamic-pituitary-adrenal (HPA) axis suppress hippocampal neurogenesis and lead to deficits in learning and memory. Glucocorticoids have played a central role in modeling this process but other factors also change with alterations in the HPA axis. Notable among these is the apparent link between pro-inflammatory cytokines and glucocorticoids. Inflammation and subsequent elevations of interleukin-1β (IL−1β) lead to the robust elevation of glucocorticoids via the HPA axis. Inflammation is also accompanied by the central production of pro-inflammatory cytokines. Among these are interleukin-6 and tumor necrosis factor-α (TNFα) which are found to be inhibitory to neurogenesis.

It is well-known that radiation is damaging to cells. Initial deposition of energy in irradiated cells occurs in the form of ionized and excited atoms or molecules distributed at random throughout the cells. It is the ionizations that cause most of the chemical changes in the vicinity of the event, by producing a positively charged or “ionized” molecule. These molecules are highly unstable and rapidly undergo chemical change to produce free radicals, atoms, or molecules containing unpaired electrons. These free radicals are extremely reactive and can lead to permanent damage of the affected molecule. As an immediate consequence of radiation damage, cells can undergo apoptosis, dying in interphase within a few hours of irradiation. Radiation damage can be acute, or can be manifested long after the initial event.

Cranial radiation therapy, a crucial treatment for brain tumors and other cancers, causes a progressive and debilitating decline in learning and memory. Cranial irradiation ablates hippocampal neurogenesis by damaging the neurogenic microenvironment. Endogenous neurogenesis is inhibited after irradiation despite the presence of neural precursor cells that retain the ability to make neurons, and neurogenesis is likewise inhibited for non-irradiated precursor cells transplanted to the irradiated hippocampus.

The investigation and development of methods to prevent this impairment in neurogenesis is of great clinical interest.

Publications

The appearance of activated microglia in the brain is a common indicator of the inflammatory process and neuroinflammation and accompanying microglial pathology, which are associated with many diseases of cognition in which memory loss features prominently, such as Alzheimer's Disease, Lewy Body Dementia, and AIDS Dementia Complex. Clinical treatment with indomethacin and other NSAIDs has been demonstrated to ameliorate the risk and progression of memory loss (Rogers et al. (1993) Neurology 43:1609-1611; (2001) N.Enql.J.Med. 345:1515-1521).

### SUMMARY OF THE INVENTION

Methods are provided for preventing defects in neurogenesis following conditions that result in neuroinflammation in the brain. The differentiation of neuronal precursor cells is shown to be adversely affected by the presence of inflammation in the brain. Among the components of inflammation, activated microglial cells are particularly harmful, and directly impair neuronal precursor cell differentiation. Such microglial cells can be resident in the brain, or can be recruited from the pool of circulating leukocytes by altered trafficking signals related to the neuroinflammatory process. Additional alterations within the neuronal precursor or stem cell microenvironment also accompany the activation of microglial cells in the brain. Such inflammatory changes in the microvasculature and other cell populations, such as astrocytes and neighboring neurons, impair the stem cell or progenitor cell's ability to generate neurons.

Methods of prevention reduce one or more of the adverse aspects of neuroinflammation. In one embodiment of the invention, general anti-inflammatory agents, e.g. NSAIDs, are administered. In another embodiment of the invention, agents are administered that block the recruitment and/or entry of circulating monocytes into the brain, including agents that antagonize chemokines, such as MCP-1. In another embodiment of the invention, agents that specifically block harmful cytokines, including IL-6; IL-1β; and TNFα; are administered. Local or systemic block of IL-6 activity is of particular interest, including administration of IL-6 blocking agents or related gp130 signaling modulators. Combinations of such therapies are also of interest.

Conditions giving rise to inflammation and subsequent changes in the stem cell signaling environment include radiation, surgery, trauma, autoimmune disease, neurodegenerative disease and other neuroinflammatory conditions. Transplantation of neuronal stem cells or other cell types intended to influence stem cell or progenitor cells, e.g. for therapeutic purposes, can also give rise to inflammation, and benefits from the methods of the invention. Administration of anti-inflammatory agents, prevents such activation of microglial cells or blocks the effect of cytokines produced by microglial cells and other cellular components of the neuroinflammatory process, such as activated astrocytes. By preventing or diminishing a loss of neurogenesis capacity, patients retain cognitive function that would otherwise be lost. In one embodiment of the invention, an improved method of cranial radiation therapy is provided, where anti-inflammatory agents are administered in conjunction with radiotherapy.

### BRIEF DESCRIPTION OF THE DRAWINGS

FIG. 1 Inflammation inhibits hippocampal neurogenesis. Lipopolysaccharide (LPS, 1 mg/Kg i.p.) was given to induce a systemic inflammatory response, followed by daily injections of BrdU for 6 days to label proliferating cells and sacrifice on the ₇^{th} day. Some rats were given the anti-inflammatory drug indomethacin twice each day (2.5 mg/kg i.p.) starting concurrently with LPS and continuing for the 1 week paradigm. (A) Schematic depicting the anatomic location of the dentate gyrus of the hippocampus within the rodent brain. The neurogenic region of the hippocampus, the granule cell layer is highlighted in red. To the right, a confocal photomicrograph shows detail of the dentate gyrus stained for the immature neuronal marker doublecortin (Dcx, red) and BrdU (proliferative cells, green). Immature neurons line the subgranule zone at the junction between the granule cell layer and the hilus of the hippocampal dentate gyrus. Scale bar, 100 μm. (B, C) Confocal micrographs of vasculature (tomato lectin, blue), BrdU-labeled cells (green) and activated microglia (ED-1, red). Proliferative cells are clustered in large groups proximal to the vasculature in naive animals (B) while clustering and proximity to the vasculature is decreased in concert with striking activation of microglia following LPS treatment (C). Scale bar, 30 μm. (D, E) BrdU-labeled newborn neurons (BrdU, green; Dcx, red) are abundant in naive animals (D) but significantly reduced following systemic LPS exposure (E). (F) Density of activated microglia (ED1+) in the granule cell layer and subgranule zone. Data are expressed as ED-1 positve cells per mm² in a 40 micron section. Systemic LPS exposure significantly increases the density of activated microglia (P<0.05; n=3); treatment with indomethacin decreases this inflammatory response. (G) Neuroinflammation induced by systemic LPS inhibits neurogenesis (P<0.05; n=3), as determined by phenotype-specific immunohistochemistry and confocal analysis. Anti-inflammatory therapy with indomethacin restores neurogenesis following LPS exposure (P<0.05; n=3). Data are expressed as the percent of non-microglial proliferating cells (BrdU+/CD11b−) that co-express doublecortin (Dcx) at the end of a one-week BrdU labeling paradigm. (H) Inflammation causes dissociation of the normal relationship between proliferating cells and the microvascualture. The average distance from the middle of a BrdU+nucleus to it nearest tomato lectin stained vessel was significantly increased in the context of inflammation (P<0.05; n=6); indomethacin restores the vascular association (P<0.05; n=3). Distance measurements were performed on 40 micron sections as measured in the x and y plane. Proliferating microglia (BrdU+/CD11b+) were excluded from the distance measurements.

FIG. 2 Activated microglia inhibit neurogenesis via soluble factors that include IL-6. (A) Co-culture of with microglia (MG), from microglia or exposure to recombinant IL-6 decreases neuronal differentiation in vitro. GFP-positive neural progenitor cells (NPCs) were induced to differentiate for 60 hrs in the presence or absence of microglia (MG) which were cultured under non-stimulated conditions (−) or stimulated with LPS for 24 hrs prior to co-culture (+). NPCs were also treated with conditioned media (CM) from these same microglial cultures, CM from activated microglia that was pre-mixed with a blocking antibody to IL-6 (αIL6) or recombinant IL-6 in the presence or absence of blocking antibody. Data were collected as the fraction of GFP-expressing cells (N PCs) that co-express the early neuronal marker doublecortin. Co-culture with unstimulated microglia (−MG) had no effect on neuronal differentiation (Student's t test; P=0.53). Co-culture with LPS-stimulated microglia decreased neurogenesis (P<0.05, relative to control or microglial co-culture) as did CM from activated but not resting microglia (Student's t test; P<0.05). A blocking antibody to IL-6 abrogates the CM effects and IL-6 alone (50 ng/ml) reproduced effects of activated microglial CM in reducing neurogenesis (P<0.05, n=3). Data are expressed as the fraction Dcx-positive cells relative to untreated control cultures. (B-D) Confocal micrographs of representative NPC cultures stained for green fluorescent protein which identifies progenitor cells (green) and neuronal double cortin (red). (B) Naive cells. (C) Cells exposed to conditioned media from activated microglia. (D) Cells exposed to IL-6 (50 ng/ml). Scale bar, 15 μm. (E) Cell fate profile following IL-6 exposure. NPCs were induced to differentiate for 60 hours in the presence of IL-6 (50 ng/ml), and the percentage of cells expressing lineage-specific markers for neurons (type III β-tubulin, βTubulin), astroctyes (glial fibrillary acidic protein, GFAP) and immature oligodendrocytes (NG2 condroitin sulfate proteoglycans). Data are expressed as the fraction of cells positive for a given marker normalized to untreated controls. IL-6 caused a significant decrease in the proportion of cells adopting a neuronal fate (P<0.05; n=3), while astrogliogenesis and oligogliogenesis were unaffected. (F) TUNEL staining in Dcx-positive cells. As in A, cultures were treated with conditioned medium from LPS-stimulated microglia or treated directly with recombinant IL-6 (50 ng/ml). TUNEL was then scored in the total population (see text) as well as within the subset of cells that had adopted a neuronal phenotype (F). Apoptosis increased significantly overall but to a larger extent in neurons relative to non-neuronal cells.

FIG. 3 Indomethacin decreases microglial inflammation following irradiation. Microglial proliferation and activation in non-irradiated (NIR) and irradiated (IR) hippocampi. Indomethacin (Ind, +/−2.5 mg/Kg) administered orally every 12 hours beginning the day before and for 2 months after irradiation. All groups received BrdU once a day for 6 days starting 4 weeks after irradiation. Animals were killed 2 months after irradiation). (A) Unbiased stereologic quantification of ED1-positive activated microglia per dentate gyrus demonstrates that indomethacin reduces the total number of activated (ED1-positive) microglia per dentate gyrus by roughly 35% (n=4 animals per group; Student's t test; P<0.05). (B,C) Examples of BrdU-labeled (red) microglia (CD11b, green) that are either negative (B) or positive for NG2 (blue, C). The NG2 epitope is known to be expressed by peripheral monocytes that are recruited into the brain during inflammation. Scale bar, 25 μm. (D) Quantification of microglia and invading peripheral monocytes in irradiated or non-irradiated animals concurrently treated with indomethacin (+/−). Irradiation caused a dramatic increase in proliferating microglia (CD11b/BrdU-double positive cells) in the granule cell layer and subgranule zone of irradiated animals relative to non-irradiated controls (n=4 animals per group; Student's t test; P<0.000001). Indomethacin had little effect on the relative fraction of BrdU-labeled cells that were microglia following radiation but significantly reduced the activation state (A) and the relative number of cells that were recruited from the periphery (NG2-positive/CD11b positive monocytes, P<0.05, Student's T test, n=4).

FIG. 4 Anti-inflammatory therapy restores neurogenesis following irradiation. Effect of indomethacin on newborn cells within the SGZ and granule cell layer. Non-irradiated NIR, white bars; irradiated, IR, black bars. Indomethacin (+/−2.5 mg/Kg) was administered orally every 12 hours beginning the day before and for 2 months after irradiation. (A) Relative proportion of proliferative cells adopting a recognized cell fate (NeuN=mature neurons; Tuj1=immature neurons; GFAP=astrocytes; NG2+/CD11b-=immature oligodendrocytes). Data are expressed as means +/− S.E.M; n=4 animals per group. Anti-inflammatory therapy with indomethacin increased the relative proportion of the proliferative cells adopting a neuronal phenotype by 2.5 fold (Student's t test; P<0.01). (B-E) Representative confocal micrographs of BrdU-labeled mature neurons (B, NeuN, green; GFAP, blue; BrdU, red); immature neurons (C, type III β tubulin, blue; NG2, green; BrdU, red); astrocytes (D, GFAP, green; NeuN, blue); and oligodendrocytes (E, NG1, green, CD11b, blue; BrdU, red). Scale bars=10 μm. (F) Increase in total number of newborn neurons per GCL+SGZ in irradiated animals treated with indomethacin. Unbiased stereologic quantification of BrdU+ cells adjusted for fraction of BrdU+ cells adopting a neuronal phenotype (NeuN+ plus Tuj1+). IR=irradiated; IR+Indo=irradiated, indomethacin treated. Anti-inflammatory therapy substantially increases the absolute number of newborn neurons per hippocampus (Student's t test; P<0.01). (G) Inflammation negatively correlates with the accumulation of new neurons. The fraction of dividing cells adopting a neuronal phenotype is inversely proportional to total number of activated microglia per dentate gyrus. Each data point represents one irradiated animal. Control irradiated animals (black diamonds), indomethacin-treated irradiated animals (gray squares).

FIG. 5. Dcx staining and pyknotic TUNEL positive nuclei in treated NPC cultures. NPC cultures were allowed to differentiate normally (A) or in the presence of IL-6 (B) or microglial conditioned medium (C). Treatment with either IL-6 or CM results in decreased Dcx staining (blue) and increased incidence of TUNEL-positive nuclei or nuclear fragments (green), many of which are also immunoreactive for Dcx. Scale bar, 20 um. D. Total RNA was collected from control cultures (“C”) or cultures treated with IL-6 (IL-6) and evaluated for the presence of IL-6 receptor transcripts by RT-PCR +/− reverse transcriptase (rt). The 67 bp PCR product is easily detectable in control cultures and appears to be unregulated following IL-6 treatment. 100 bp ladder (M).

FIG. 6. Orthogonal projections of NG2-associated microglia in the irradiated brain. Microglia in irradiated or LPS treated animals frequently co-localize with NG2 proteoglycan. It is thought that NG2-positive, CD11b-positive cells are peripheral monocytes recruited to the brain following injury. However, microglia are also known to promote myelination and colocalization of NG2 with CD11b can also occur when oligodendrocytes envelope activated microglia. Shown are two examples of NG2 co-localized with CD11b staining. In A, the NG2 staining (blue) is associated with the CD11b-positive microglial cell (green, BrdU in red) but appears to originate from neighboring oligodendrocyte processes. In B, the NG2 staining and CD11b staining tightly co-localize to the cytoplasm and membrane of an individual microglial cell. Quantification of NG2-positive microglia in FIG. 3D excluded microglia where NG2 immunoreactivity was clearly associated with an enveloping oligodendrocyte process. Scale bar, 20 μm.

FIG. 7. Inflammation may inhibit neurogenesis by multiple mechanisms. Neural stem cells (SC) can differentiate into neurons, oligodendrocytes or astrocytes. Inflammation may disrupt neuronal differentiation by directly inhibiting neuronal fate choice and differentiation resulting in a diversion of cell fate into glial lineages (dashed arrows). This may be via gp130 mediated activation of Notch pathway genes, or indirectly by altering the interaction of neural progenitor cells with other cells of the local microenvironment such as cells of the vasculature within the subgranule zone (see FIG. 1 B, C, H). The radiation-induced peripheral monocyte recruitment and its inhibition with indomethacin provide clear evidence that endothelial cell status is significantly modulated in inflammation and inflammatory blockade. Finally, inflammation is known to modulate the hypothalamic-pituitary-adrenal axis and a concurrent elevation in circulating glucocorticoid levels would feed back into the neurogenic regulatory mechanisms to suppress hippocampal neurogenesis.

FIG. 8. Hippocampal neurogenesis following radiation in adult MCP-1 deficient mice. Animals were treated with a single dose of 10 Gy cranial x-irradiation and then allowed to recover for one month. BrdU was then administered once each day for 6 days (beginning week 5 after irradiation) and then animals allowed to survive for an additional three weeks. Hippocampal formations were then evaluated for surviving newborn neurons 8 weeks after irradiation (one month after the initial BrdU injection). A. Total BrdU labeled cells per dentate gyrus of the hippocampus. Irradiation severely inhibits accumulation of BrdU labeled cells in wild type animals but has no significant impact on MCP-1 null animals. B. Fraction of BrdU cells adopting a neuronal phenotype (NeuN or doublecortin, Dcx positive). As for proliferative activity, the production and survival of neurons is severely attenuated in irradiated wild type mice but is completely unaffected in MCP-1 null animals. C. The total number of new born neurons detectable in the hippocampus can be derived by multiplying the fraction of new born cells that are neurons by the total number of newborn cells. As previously observed in rats, wild type mice show a >75% decrease in net neurogenesis after a single dose of 10Gy x-irradiation. Neurogenesis in MCP-1 null animals is unaffected by x-irradiation.

### DETAILED DESCRIPTION OF THE EMBODIMENTS

Methods are provided for protecting an individual from adverse long-term effects of deficits in neurogenesis that can follow acute or chronic neuroinflammation. Inflammatory blockade with a general or specific anti-inflammatory drug prevents a loss of neurogenesis capacity after, for example, cranial irradiation, or other neuroinflammatory conditions, including naturally occurring and induced episodes of inflammation. This inflammatory blockade reduces the direct effects of activated microglia on neural precursor cells and restores the cytokine-interrupted signaling by neighboring cells that is necessary to support neurogenesis. These cells include the essential cellular components of the stem/progenitor cell local microenvironment, e.g. microvascular endothelium, smooth muscle, astrocytes and neurons. These findings have broad implications for a variety of diseases of cognition involving neuroinflammation, inflammatory cytokines and stem cell/precursor cell dysfunction.

In one embodiment of the invention, inflammatory blockade is coupled with cranial radiation therapy. Cranial radiation can cause a progressive decline in cognition that is linked to long-term ablation of hippocampal neurogenesis. Robust microglial inflammation accompanies irradiation-induced, microenvironmental failure and mediates the neurogenic failure. Cranial irradiation increases the production of pro-inflammatory cytokines and chemokines in the brains of both mice and men, in particular the production of MCP-1; IL-6; and TNF-α.

The methods of the invention are useful in prevention of cognitive radiation damage from a variety of sources of ionizing radiation, including X-rays, gamma-rays, beta radiation and alpha radiation. Such radiation may result from exposure to nuclear fusion or fission materials, e.g. nuclear waste, nuclear weapons, or nuclear power plants, from intentional or unintentional exposure to radiation, e.g. X-rays, gamma rays, etc. for medical or other purposes.

The methods are also useful in preventing cognitive damage that results from neuroinflammation, immune cytokines and precursor cell dysfunction in a variety of diseases including Alzheimer's disease, Parkinson's disease, Huntington's disease, lysosomal storage disorders involving inflammatory response, multiple sclerosis or other auto-immune disease, depression, bipolar disorder, or Cushing's disease and other iatrogenic hyperglucocorticoid “Cushingoid” states.

Additional diseases benefit from these methods due to their known recruitment of immune-mediated processes and accompanying deficits in cognition, in which defects in neurogenesis are implicated. These include Lewy Body dementia, Frontotemporal dementia/Pick's disease, AIDS dementia complex, dementia puligistica and chronic cognitive dysfunction following head trauma, prion-associated dementia such as Creutzfeldt-Jacob disease, cognitive dysfunction following chronic seizure disorders or an episode of, status epilepticus, cognitive dysfunction following encephalitis or meningitis, amyotrophic lateral sclerosis (ALS)/parkinsonian/ dementia complex of Guam.

In one embodiment, the methods are also useful for attenuating the inflammatory effects on neurogenesis following acute injury, such as traumatic injury, ischemia, cerebral hemorrhage, or stroke. In another embodiment, the methods are useful for attenuating the effects of pre- or peri-natal ischemia/hemorrhage associated with the developmental dysregulation of stem/progenitor cells in early life.

The methods of the invention find use in the treatment of post-trauma or post-surgical control of brain inflammation or other inflammatory processes, which are currently treated with exogenous corticosteroids, as corticosteroids intrinsically inhibit neurogenesis and accentuate the already detrimental effects of neuroinflammation on neurogenesis. In the stress/depression context, post-traumatic stress disorder is expected to have a cytokine/inflammation mediated dysfunction, treatable by the methods of the invention.

The methods are used for augmenting abortive neurogenesis that occurs in response to surgical interventions, injury, or disease but which is attenuated by virtue of an accompanying immune response.

The methods of the invention find use in minimizing the negative influence of inflammation in cell or tissue transplantation to the central nervous system, where such grafts include neural progenitors such as those found in fetal tissues, neural stem cells, embryonic stem cells or other cells and tissues contemplated for neural repair or augmentation. Neural stem/progenitor cells have been described in the art, and their use in a variety of therapeutic protocols has been widely discussed. For example, interalia, U.S. Pat. No. 6,638,501, Bjornson et al.; U.S. Pat. No. 6,541,255, Snyder et al.; U.S. Pat. No. 6,498,018, Carpenter; U.S. patent application Ser. No. 20020012903, Goldman et al.; Palmer et al. (2001) Nature 411(6833):42-3; Palmer et al. (1997) Mol Cell Neurosci. 8(6):389-404; Svendsen et al. (1997) Exp. Neurol. 148(1):135-46 and Shihabuddin (1999) Mol Med Today. 5(11):474-80; each herein specifically incorporated by reference.

Neural stem and progenitor cells can participate in aspects of normal development, including migration along well-established migratory pathways to disseminated CNS regions, differentiation into multiple developmentally- and regionally-appropriate cell types in response to microenvironmental cues, and non-disruptive, non-tumorigenic interspersion with host progenitors and their progeny. Human NSCs are capable of expressing foreign transgenes in vivo in these disseminated locations. A such, these cells find use in the treatment of a variety of conditions, including traumatic injury to the spinal cord, brain, and peripheral nervous system; treatment of degenerative disorders including Alzheimer's disease, Huntington's disease, Parkinson's disease; affective disorders including major depression; stroke; and the like. During the physical manipulation involved in transplantation, physical damage can cause neuroinflammation, which then limits the ability of the transplanted cells to thrive in the recipient environment. By administering anti-inflammatory agents, the deleterious effects of neuroinflammation are reduced, providing enhanced engraftment and neuron growth.

The methods find use in developing ligand-targeted compound or gene delivery systems where detection, diagnosis, and clinical monitoring of immune-mediated stem/progenitor cell dysfunction is desired. Such strategies include the use of anti-inflammatory agents to validate the predictive nature of the detection method(s) in correcting or modifying stem/progenitor cell function.

The similarities between neural stem cells in the central and peripheral nervous system also indicate that these methods are useful in augmenting neural tissue repair in the peripheral nervous system, where local inflammation may prevent optimum healing or restoration of innervation by virtue of neural stem/progenitor cell dysfunction. Such diseases or injury may include nerve injury due to trauma, surgery, cancer, or immune disease such as multiple sclerosis, ALS, or other motor neuron disease where endogenous or grafted progenitor/stem cells are influenced by immune mechanisms.

General anti-inflammatory agents useful in protection of neurogenesis include those drugs generally classified as nonsteroidal anti-inflammatory drugs (NSAIDs). Byway of example and not limitation, NSAIDs useful in the practice of the invention include fenoprofen calcium, nalfon, flurbiprofen, Ansaid, ibuprofen, ketoprofen, naproxen, anaprox, aflaxen, oxaprozin, diclofenac sodium, diclofenac potassium, cataflam, etodolac, indomethacin, ketorolac tromethamine, nabumetone, sulindac, tolmetin sodium, fenamates, meclofenamate sodium, mefenamic acid, piroxicam, salicylic acid, diflunisal, aspirin, oxyphenbutazone, and phenylbutazone.

A subpopulation of microglia involved in deleterious inflammation are peripheral blood monocytes/microglia, which contribute to chronic neuroinflammatory lesions within the brain by entry across the blood brain barrier resulting from altered patterns of leukocyte trafficking. The altered pattems result from changes in chemokine and/or leukocyte adhesion molecule signaling. In addition to general anti-inflammatory agents, specific agents, e.g. those known in the art, that interrupt the recruitment of leukocytes to the brain are of interest as therapeutic agents. Compound screening can also be performed to identify agents that specifically interfere with the trans-endothelial migration of monocytes/microglia. Anti-inflammatory agents are particularly effective at decreasing this subpopulation of infiltrating, proliferating peripheral monocytes.

Various adhesion and chemokines molecules have been implicated in leukocyte trafficking to the brain. Vries et al. (2002) J Immunol. 168(11):5832-9 states that signal-regulatory protein (SIRP)alpha-CD47 increases monocyte transmigration across brain ECs. CD47 is expressed on cerebral endothelium, while SIRPalpha and CD47 are expressed on monocytes. James et al. (2003) J Immunol. 170(1):520-7 found that blockade of alpha₄ integrin or VCAM-1 inhibited leukocyte rolling and adhesion to the cerebral vasculature. The chemokines monocyte chemoattractant protein-1 and IL-8 have also been implicated in brain leukocyte trafficking, as well as MCP-1 (CCL2), SDF-1, MIP-1 alpha (CCL3), MIP-1 beta, RANTES (CCL5), eotaxin (CCL11), and MIP-2.

As shown in the examples, in the absence of MCP-1 the deleterious effects associated with events that create neuroinflammation are abrogated. Therefore, agents of particular interest include antagonists and inhibitors of MCP-1. Many such agents have been described in the art, and may find use in the methods of the invention. Such agents include small molecules; polypeptides; antisense and siRNA; and the like. Small molecule antagonists include, without limitation, 5-(((S)-2,2-dimethylcyclopropanecarbonyl)amino)phenoxy)pyridine (APC0576, described by Yuzawa et al. (2003) Transplantation 75:1463-1468); 2-methyl-2-[[1-(phenylmethyl)-1H-indazol3yl]methoxy] propanoic acid (Bindarit, described by Sironi et al. (1999) European Cytokine Network 10:437-442); 17 β-estradiol (described by Kanda et al. (2003) J Invest Dermatol. 120(6): 1058-66); trans-3,4-dichloro-N-methyl-N [2-(1-pyrolidinyl)cyclohexyl] benzeneacetamide methanesulfonate (U50,488, described by Sheng et al. (2003) Biochem Pharmacol. 65(1):9-14); doxazosin, described by Kintscher et al. (2001) J Cardiovasc Pharmacol. 37(5):532-9). Polypeptide antagonists include, without limitation, NH(2)-terminal-truncated MCP-1 (described by Hasegawa et al. (2003) Arthritis Rheum. 48(9):2555-66); 7ND (described by Shimizu et al. (2003) J Am Soc Nephrol. 14(6):1496-505); eotaxin-3 (described by Ogilvie et al. (2003) Blood 102(3):789-94). RNA based antagonists include high affinity aptamers, e.g. ADR7 and ADR22 as described by Rhodes et al. (2001) FEBS Lett. 506(2):85-90.

Other agents of interest are targeted to specifically block the activity of interleukin-6 (IL-6), as IL-6 is shown herein to suppress hippocampal neurogenesis. IL-6 is a pleiotropic cytokine with a wide range of biological functions. These IL-6 functions are mediated through a receptor system composed of two different molecules on the cell surface. One is an IL-6 binding molecule, IL-6 receptor (IL-6R), and the other is a common signal transducer for IL-6 family cytokines, gp130. Following the binding of IL-6 with IL-6R, the IL-6 signal is transduced into the cells through gp130, which binds the complex of IL-6 and IL-6R. Two types of IL-6R molecules exist in vivo. One is the above-mentioned membrane-bound IL-6R (80 kd), and the other is a soluble form of IL-6R (50 kd, slL-6R), which is secreted into the serum by the alternative splicing of mRNA and the enzymatic cleavage of 80 kd IL-6R on the cell surface. This slL-6R can also mediate the IL-6 signal into cells via gp130 in the same way as IL-6R, so that IL-6R functions as an agonist to the IL-6 signal transduction.

Functional blocking of IL-6 activity may be achieved by inhibiting IL-6 production; neutralizing IL-6 protein; blocking IL-6 binding to IL-6R; blocking IL-6/IL-6R complex binding to gp130 molecule, suppressing IL-6R and/or gp130 expression; or blocking intracytoplasmic signal transduction through gp130. In a preferred embodiment, a specific binding agent is used to block IL-6 binding to IL-6R. Humanized antibodies that bind to the IL-6R are known in the art (Yoshizaki et al. (1998) Springer Semin Immunopathol 20:247).

In addition to IL-6; other cytokines have been shown to act on the vasculature and/or neuronal stem cells and to reduce neurogenesis. Such cytokines include IL-1β, and TNFα. IL-1beta is a pro-inflammatory cytokine that appears in brain and cerebrospinal fluid following peripheral immune challenges and central infections or injury. The cytokine has a systemic effect, and may additionally have a specific effect on vascular endothelial cells. IL-1b has also been credited with inducing expression of monocyte chemoattractant protein-1 (mcp-1) and intercellular adhesion molecule-1 (ICAM-1). It may trigger a targeted leukocyte emigration and widespread glial activation (see Proescholdt et al. (2002) Neuroscience 112(3):731-49).

Antagonists that block the activity of these cytokines may also find use in the methods of the invention. Many agents that block activity of TNFα are described in the art, for example see U.S. patent application Ser. No. 20010022978; U.S. Pat. No. 6,537,540; etc. Both antibody and small molecules inhibitors of IL-1 b have been described, for example see U.S. Pat. Nos. 6,541,623; 6,511,665; 6,337,072; 6,133,274; etc.

Therapeutic formulations of general and specific anti-inflammatory agents, including MCP-1 and IL-6 blocking agents, are provided. In one aspect of the invention, the anti-inflammatory agent is administered to individuals having an increased chance of cranial radiation toxicity. The formulations find use as protective agents, for example, in cancer patients treated with ionizing radiation. The agent can be administered locally or systemically against anticipated radiation exposure, e.g. radiation therapy or exposure resulting from workplace radiation, military exposure, and the like. In another embodiment, the agent is administered locally or systemically immediately following accidental or unintentional exposure.

The compounds of the present invention are administered at a dosage that protects the cell population while minimizing any side-effects. It is contemplated that the composition will be obtained and used under the guidance of a physician for in vivo use. The dosage of the therapeutic formulation will vary widely, depending upon the nature of the disease, the frequency of administration, the manner of administration, the clearance of the agent from the host, and the like.

In one embodiment of the invention, the anti-inflammatory agent is administered in conjunction with cranial radiation treatment of a tumor. Tumors for which cranial radiation may be indicated include primary brain tumors, tumors metastatic to the brain, central nervous system involvement of leukemias and lymphomas, and head and neck cancers, or other cancers or neoplasias that involve radiation treatment fields that include the central nervous system. Several methods of interest include the combination administering an anti-inflammatory agent in conjunction with whole body irradiation as administered in bone marrow transplant, cranial irradiation as used to treat diffuse tumors of the head and neck, focal irradiation as delivered by the CyberKnive or equivalent shaped field or restricted beam delivery system such as a proton beam, GliaSite radiation, which irradiates cancerous cells from within the tumor cavity, or ligand-targeted delivery of radioactive agents such as antibody-linked or synthetic molecule linked radio-ablative compounds.

Brain tumors are classified according to the kind of cell from which the tumor seems to originate. Diffuse, fibrillary astrocytomas are the most common type of primary brain tumor in adults. These tumors are divided histopathologically into three grades of malignancy: World Health Organization (WHO) grade II astrocytoma, WHO grade III anaplastic astrocytoma and WHO grade IV glioblastoma multiforme (GBM). Biological subsets of primary brain tumors include adrenocartical carcinoma; brain stem gliomas; pleomorphic xanthoastrocytoma (PXA); pilocytic astrocytoma; subependymal giant cell astrocytomas; desmoplastic cerebral astrocytoma of infancy (DCAI); desmoplastic infantile ganglioglioma; oligodendrogliomas; oligoastrocytomas (mixed gliomas); ependymomas; supratentorial intraventricular tumors; benign cerebellopontine angle tumors; medulloblastomas; meningiomas; schwannomas; hemangioblastomas; and hemangiopericytomas. Brain metastases are one of the most common sites of systemic spread from solid tumors. Metastatic cancers of the brain include, without limitation, non-small cell lung cancer; breast cancer; melanoma; renal and colon cancers; and the like. Primary central nervous system (CNS) lymphoma is a malignant neoplasm of lymphocytic derivation that is localized to the nervous system. The incidence of these tumors is rising relative to other brain lesions due to the occurrence of primary lymphoma in AIDS and transplant patients. Most common supratentorial locations are the frontal lobes, then deep nuclei and periventricular zone.

Surgery is often used in the treatment of brain tumors to remove or reduce as much of its bulk as possible. By reducing the size of tumor mass, radiotherapy can be more effective. Stereotaxy is a useful adjunct to surgery and radiotherapy (stereotactic radiotherapy). The compositions of the invention may be administered using any medically appropriate procedure, e.g., intravascular (intravenous, intraarterial, intracapillary) administration, injection into the cerebrospinal fluid, intracavity or direct injection in the tumor. Intrathecal administration maybe carried out through the use of an Ommaya reservoir, in accordance with known techniques. (F. Balis et al., Am J. Pediatr. Hematol. Oncol. 11, 74, 76 (1989).

The effective amount of a therapeutic composition to be given to a particular patient will depend on a variety of factors, several of which will be different from patient to patient. Utilizing ordinary skill, the competent clinician will be able to optimize the dosage of a particular therapeutic or imaging composition in the course of routine clinical trials.

Anti-inflammatory agents can be incorporated into a variety of formulations for therapeutic administration by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation.

One strategy for drug delivery through the blood brain barrier (BBB) entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can be co-administered with the therapeutic compositions of the invention when the compositions are administered by intravascular injection. Other strategies to go through the BBB may entail the use of endogenous transport systems, including carrier-mediated transporters such as glucose and amino acid carriers, receptor-mediated transcytosis for insulin or transferrin, and active efflux transporters such as p-glycoprotein. Active transport moieties may also be conjugated to the therapeutic or imaging compounds for use in the invention to facilitate transport across the epithelial wall of the blood vessel. Alternatively, drug delivery behind the BBB is by intrathecal delivery of therapeutics or imaging agents directly to the cranium, as through an Ommaya reservoir.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The active ingredient, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The compositions of the invention may be administered using any medically appropriate procedure, e.g., intravascular (intravenous, intraarterial, intracapillary) administration, injection into the cerebrospinal fluid, intracavity or direct injection in the tumor. Intrathecal administration maybe carried out through the use of an Ommaya reservoir, in accordance with known techniques. (F. Balis et al., Am J. Pediatr. Hematol. Oncol. 11, 74, 76(1989). For the imaging compositions of the invention, administration via intravascular injection is preferred for pre-operative visualization of the tumor. Post-operative visualization or visualization concurrent with an operation may be through intrathecal or intracavity administration, as through an Ommaya reservoir, or also by intravascular administration.

Where the therapeutic agents are administered in combination with treatment of brain tumors, one method for administration of the therapeutic compositions of the invention is by deposition into or near the tumor by any suitable technique, such as by direct injection (aided by stereotaxic positioning of an injection syringe, if necessary) or by placing the tip of an Ommaya reservoir into a cavity, or cyst, for administration. Alternatively, a convection-enhanced delivery catheter may be implanted directly into the tumor mass, into a natural or surgically created cyst, or into the normal brain mass. Such convection-enhanced pharmaceutical composition delivery devices greatly improve the diffusion of the composition throughout the brain mass. The implanted catheters of these delivery devices utilize high-flow microinfusion (with flow rates in the range of about 0.5 to 15.0 μl/minute), rather than diffusive flow, to deliver the therapeutic composition to the brain and/or tumor mass. Such devices are described in U.S. Pat. No. 5,720,720, incorporated fully herein by reference.

The effective amount of a therapeutic composition to be given to a particular patient will depend on a variety of factors, several of which will be different from patient to patient. A competent clinician will be able to determine an effective amount of a therapeutic agent to administer to a patient to prevent or decrease ongoing neuroinflammation. Dosage of the agent will depend on the treatment, route of administration, the nature of the therapeutics, sensitivity of the patient to the therapeutics, etc. Utilizing LD₅₀ animal data, and other information, a clinician can determine the maximum safe dose for an individual, depending on the route of administration. Utilizing ordinary skill, the competent clinician will be able to optimize the dosage of a particular therapeutic composition in the course of routine clinical trials. The compositions can be administered to the subject in a series of more than one administration. For therapeutic compositions, regular periodic administration will sometimes be required, or may be desirable. Therapeutic regimens will vary with the agent, e.g. an NSAID such as indomethacin may be taken for extended periods of time on a daily or semi-daily basis, while more selective agents, such as antagonists of MCP-1, may be administered for more defined time courses, e.g. one, two three or more days, one or more weeks, one or more months, etc., taken daily, semi-daily, semi-weekly, weekly, etc.

Formulations may be optimized for retention and stabilization in the brain. When the agent is administered into the cranial compartment, it is desirable for the agent to be retained in the compartment, and not to diffuse or otherwise cross the blood brain barrier. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the agent in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the subject invention. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

The methods are also useful in animal models or in vitro models for disease where drugs or therapies aimed at minimizing the negative influence of inflammation on neural stem/progenitor cell function can be discovered or optimized. Additional strategies for which this method may be useful include use for the development of viral vectors or synthetic gene delivery systems where the goals are to modify immune mechanisms and inflammatory effects on stem/progenitor cells. Such models would include genetic manipulation of cells or tissues with the result of minimizing or modifying inflammatory effects on neuroprogenitor/stem cell function.

Models of interest may include, without limitation, the use of animals and cells that have been genetically altered in the expression of pro-inflammatory chemokines and cytokines, e.g. knock-outs and knock-ins of MCP-1; IL-6; TNF-α; etc. In vitro models of interest include cultures and co-cultures in which one or more of astrocytes; microglial cells; neural progenitors; and vascular cells, e.g. endothelial cells, smooth muscle cells, etc.; are present, where the cells may be wild-type or genetically altered as described above. Such cultures find use in determining the effectiveness of candidate therapies and agents in reducing neural inflammation; in the screening of cell-cell interactions, and the like.

An embodiment of interest is the screening of candidate agents for the ability to downregulate or inhibit proinflammatory activity of neural cells. Such compound screening may be performed using an in vitro model, a genetically altered cell or animal, or purified protein corresponding to polypeptides identified herein as involved in the damaging effects of neuroinflammation, e.g. MCP-1; IL-6; TNF-α, etc. Of particular interest are screening assays for agents that have a low toxicity for normal human cells. A wide variety of assays may be used for this purpose.

For example, cell cultures modeling the interaction between neural progenitors and astrocytes may be exposed to inflammatory stimulus, such as LPS; exogenous cytokines, and the like, and the effect on neural progenitors monitored by growth, developmental commitment, expression of markers, phenotype, and the like. The cultures may include other cells, for example microglial cells. Candidate compounds are added to the cell cultures, and the effect in counteracting adverse effects of inflammation determined. As the chemokine MCP-1 is known to mediate certain of these effects, cells deficient, or alternatively constitutively expressing, MCP-1 may find use in such assays, particularly where microglial cells are present. Alternatively, cells, e.g. astrocytes, or co-cultures comprising such cells, may be used to analyze compounds for an ability to inhibit expression of MCP-1.

The term “agent” as used herein describes any molecule, e.g. protein or pharmaceutical, with the capability of inhibiting the adverse effects of neuroinflammation. It may not be required that the agent prevent inflammation, so long as the damaging effect on neural progenitors is inhibited.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Generally a plurality of assay mixtures are run in parallel with different agent concentrations to obtain a differential response to the various concentrations. Typically one of these concentrations serves as a negative control, i.e. at zero concentration or below the level of detection.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example.

Libraries of candidate compounds can also be prepared by rational design. (See generally, Cho et al., Pac. Symp. Biocompat. 305-16, 1998); Sun et al., J. Comput. Aided Mol. Des. 12:597-604, 1998); each incorporated herein by reference in their entirety). For example, libraries of phosphatase inhibitors can be prepared by syntheses of combinatorial chemical libraries (see generally DeWitt et al., Proc. Nat. Acad. Sci. USA 90:6909-13, 1993; International Patent Publication WO 94/08051; Baum, Chem. & Eng. News, 72:20-25,1994; Burbaum et al., Proc. Nat Acad. Sci. USA 92:6027-31, 1995; Baldwin et al., J. Am. Chem. Soc. 117:5588-89, 1995; Nestler et al., J. Org. Chem. 59:4723-24, 1994; Borehardt et al., J. Am. Chem. Soc. 116:373-74, 1994; Ohlmeyer et al., Proc. Nat. Acad. Sci. USA 90:10922-26, all of which are incorporated by reference herein in their entirety.)

A “combinatorial library” is a collection of compounds in which the compounds comprising the collection are composed of one or more types of subunits. Methods of making combinatorial libraries are known in the art, and include the following: U.S. Pat. Nos. 5,958,792; 5,807,683; 6,004,617; 6,077,954; which are incorporated by reference herein. The subunits can be selected from natural or unnatural moieties. The compounds of the combinatorial library differ in one or more ways with respect to the number, order, type or types of modifications made to one or more of the subunits comprising the compounds. Alternatively, a combinatorial library may refer to a collection of “core molecules” which vary as to the number, type or position of R groups they contain and/or the identity of molecules composing the core molecule. The collection of compounds is generated in a systematic way. Any method of systematically generating a collection of compounds differing from each other in one or more of the ways set forth above is a combinatorial library.

A combinatorial library can be synthesized on a solid support from one or more solid phase-bound resin starting materials. The library can contain five (5) or more, preferably ten (10) or more, organic molecules that are different from each other. Each of the different molecules is present in a detectable amount. The actual amounts of each different molecule needed so that its presence can be determined can vary due to the actual procedures used and can change as the technologies for isolation, detection and analysis advance. When the molecules are present in substantially equal molar amounts, an amount of 100 picomoles or more can be detected. Preferred libraries comprise substantially equal molar amounts of each desired reaction product and do not include relatively large or small amounts of any given molecules so that the presence of such molecules dominates or is completely suppressed in any assay.

Combinatorial libraries are generally prepared by derivatizing a starting compound onto a solid-phase support (such as a bead). In general, the solid support has a commercially available resin attached, such as a Rink or Merrifield Resin. After attachment of the starting compound, substituents are attached to the starting compound. Substituents are added to the starting compound, and can be varied by providing a mixture of reactants comprising the substituents. Examples of suitable substituents include, but are not limited to, hydrocarbon substituents, e.g. aliphatic, alicyclic substituents, aromatic, aliphatic and alicyclic-substituted aromatic nuclei, and the like, as well as cyclic substituents; substituted hydrocarbon substituents, that is, those substituents containing nonhydrocarbon radicals which do not alter the predominantly hydrocarbon substituent (e.g., halo (especially chloro and fluoro), alkoxy, mercapto, alkylmercapto, nitro, nitroso, sulfoxy, and the like); and hetero substituents, that is, substituents which, while having predominantly hydrocarbyl character, contain other than carbon atoms. Suitable heteroatoms include, for example, sulfur, oxygen, nitrogen, and such substituents as pyridyl, furanyl, thiophenyl, imidazolyl, and the like. Heteroatoms, and typically no more than one, can be present for each carbon atom in the hydrocarbon-based substituents. Alternatively, there can be no such radicals or heteroatoms in the hydrocarbon-based substituent and, therefore, the substituent can be purely hydrocarbon.

Compounds that are initially identified by any screening methods can be further tested to validate the apparent activity. The basic format of such methods involves administering a lead compound identified during an initial screen to an animal that serves as a model for humans and then determining the effects of preventing cognitive damage resulting from neuroinflammation. The animal models utilized in validation studies generally are mammals. Specific examples of suitable animals include, but are not limited to, primates, mice, and rats.

### EXPERIMENTAL

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Example 1

Cranial radiation therapy causes a progressive decline in cognitive function that is linked to impaired neurogenesis. Chronic inflammation accompanies radiation injury, suggesting that inflammatory processes may contribute to neural stem cell dysfunction. The following data demonstrate that neuroinflammation alone inhibits neurogenesis and that inflammatory blockade with indomethacin, a common non-steroidal anti-inflammatory drug, restores neurogenesis following endotoxin-induced inflammation and augments neurogenesis following cranial irradiation.

To determine the effects of inflammation on adult hippocampal neurogenesis, bacterial lipopolysaccharide (LPS) iwas injected nto adult female rats to induce systemic inflammation. The intraperitoneal administration of LPS causes a peripheral inflammatory cascade that is transduced to the brain via IL-1β from the cerebral vasculature and causes a strong upregulation of central pro-inflammatory cytokine production. Following LPS exposure, rats were treated systemically with bromo-deoxyuridine (BrdU) for 6 days to label proliferating cells within the hippocampus. Animals were then sacrificed on the ⁷th day. The fate of the BrdU-labeled, proliferative cells was analyzed with immuno-fluorescent staining and confocal microscopy.

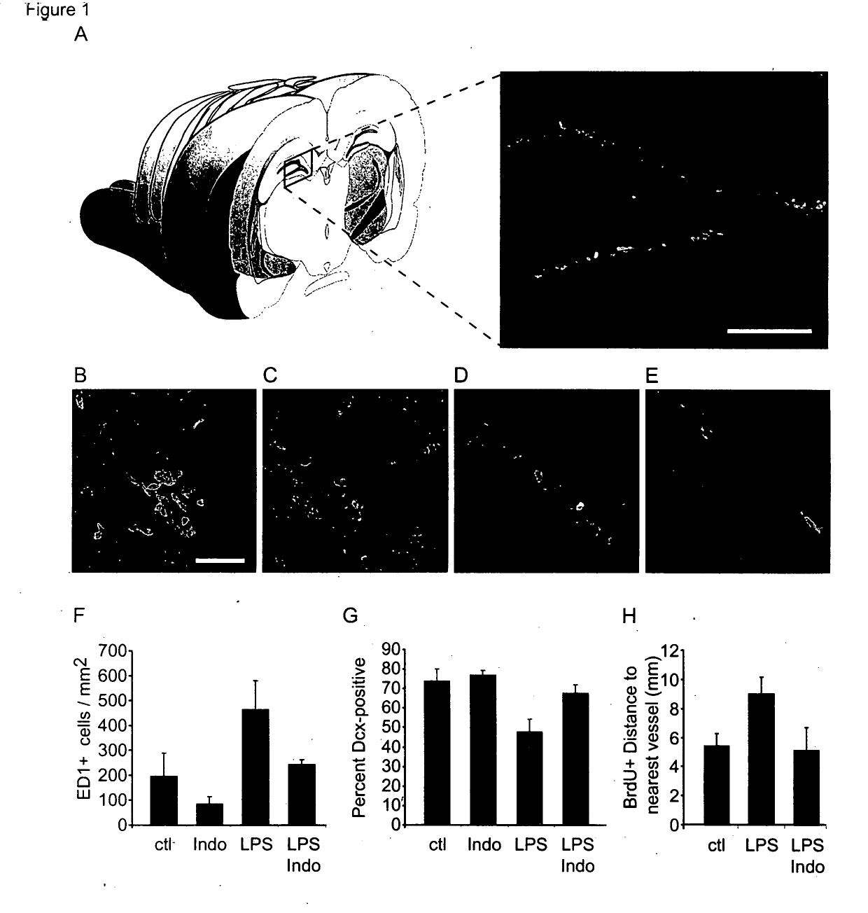

Using confocal analysis, it was found that peripheral LPS exposure resulted in a 240% increase in the density of activated microglia (CD68/ ED1-positive) in the dentate gyrus (FIG. 1A-C, F). In normal animals, few ED1-positive cells are found. The neuroinflammation achieved in the LPS paradigm was accompanied by a failure to recruit proliferation within the perivascular space, as indicated by an increase in the average distance of dividing cells (FIG. 1B, C, H) as well as a 35% decrease in hippocampal neurogenesis (FIG. 1D, E, G), as determined by the proportion of non-microglial BrdU+ proliferative cells that co-express the early neuronal marker doublecortin (Dcx).

Inflammation in the central nervous system is effectively managed using steroidal anti-inflammatory drugs, yet it is clearly demonstrated in rodents that corticosteroids are potent inhibitors of neurogenesis and their use in the context of augmenting neurogenesis would be strongly contra-indicated. To determine if inflammatory effects could be countered pharmacologically, animals were treated concurrentlywith a single dose of intraperitoneal LPS and daily doses of the non-steroidal anti-inflammatory drug (NSAID) indomethacin (2.5 mg / kg, i.p., twice each day). The effect of peripheral LPS exposure on neurogenesis was completely blocked by systemic treatment with indomethacin while indomethacin alone had no effect on neurogenesis in control animals (FIG. 1G, H).

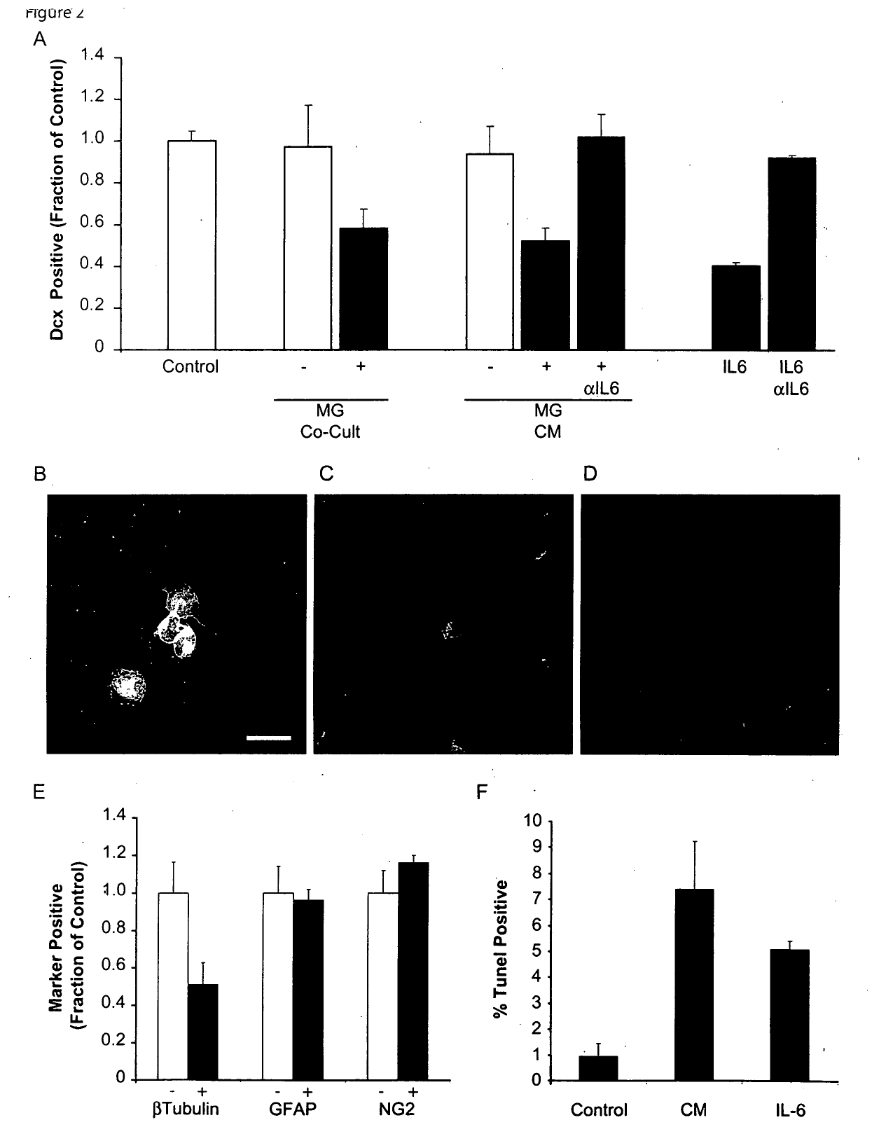

Neuroinflammation could inhibit neurogenesis by a variety of mechanisms, including stimulation of the HPA axis with subsequent elevation of gluccocorticoids, alterations in the relationships between progenitor cells and cells of the neuro-vasculature, or direct effects of activated microglia on the precursor cells. To determine the extent to which microglial activation might directly affect neural stem/progenitor cells, microglia were stimulated in vitro with LPS. LPS is a potent activator of microglia and up-regulates the elaboration of pro-inflammatory cytokines, including IL-6 and TNF-α. LPS-stimulated or resting microglia were then co-cultured with normal neural stem cells from the hippocampus under conditions that typically stimulate the differentiation of 30 to 40% of the progenitor cells into immature Dcx-expressing neurons (normalized to a value of 1 in FIG. 2A, control). Neurogenesis in the presence of microglia was assessed as the increase or decrease in Dcx-expressing cells relative to control. Co-culture with activated, but not resting, microglia decreased in vitro neurogenesis to approximately half of control levels (FIG. 2A). LPS added directly to precursor cells had no effect on neurogenesis.

To determine if this effect was due to soluble factors or due to cell-cell contact, hippocampal precursor cells were differentiated in the presence of media pre-conditioned by resting or activated microglia. A similar decrease in neurogenesis was found when precursor cells were exposed to the conditioned medium (CM) from activated microglia (FIG. 2A, C), indicating that activated microglia produce soluble anti-neurogenic factors.

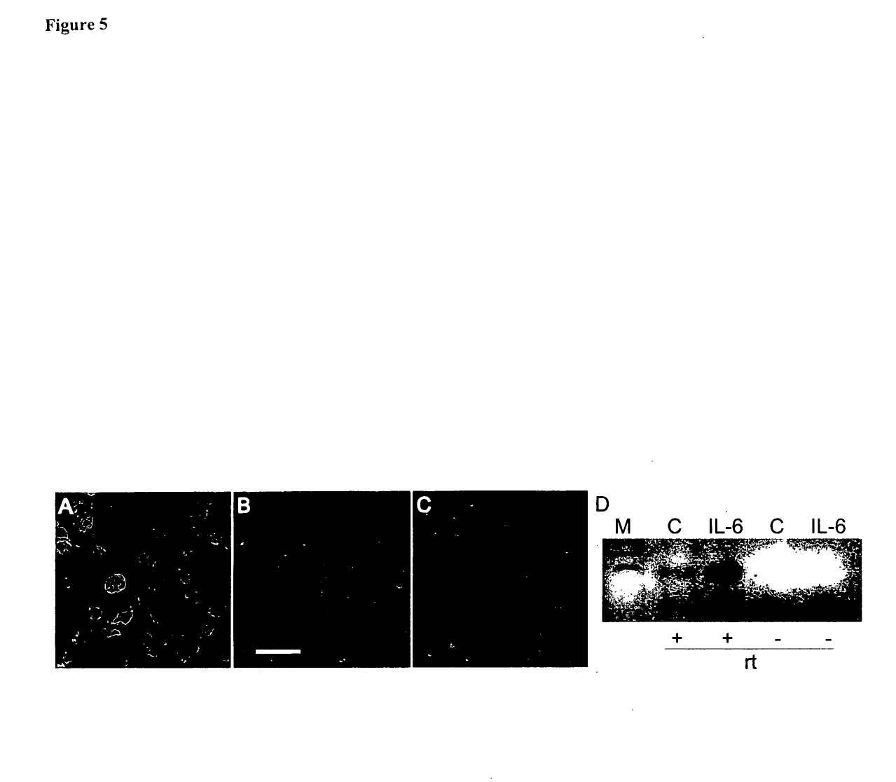

Activated microglia produce the potent pro-inflammatory cytokines interleukin-1β (IL-1β), tumor necrosis factor-α (TNF-α), interferon-γ, (INF-γ) and interleukin-6 (IL-6). Progenitor cells were allowed to differentiate in the presence of each cytokine and the relative expression of Dcx was scored after 60 hours. Exposure to recombinant IL-6 (50ng/ml) (FIG. 2A, D, E ) or to TNF-α (20ng/ml) decreased in vitro neurogenesis by approximately 50% while the effects of IL-1β or INF-γ were not significant. Addition of neutralizing anti-IL-6 antibody to CM from activated microglia was able to fully restore in vitro neurogenesis (FIG. 2A). This implicated IL-6 as a key inhibitor of neurogenesis in microglial CM. Although recombinant TNFα also suppressed neurogenesis, IL-6 blockade alone appeared sufficient to restore neurogenesis in the presence of microglial CM. In contrast to neurogenesis, gliogenesis was unaffected by IL-6 exposure as indicated by the lack of change in the number of cells expressing the astrocyte (glial fibrillary acidic protein, GFAP) or early oligodendrocyte (NG2) markers relative to control cultures (FIG. 2E). The hippocampal precursors used in this study do express the IL-6 receptor, as confirmed by RT-PCR (FIG. 5).

TUNEL labeling was used to determine the potential effects of microglial CM or IL-6 on cell death. Microglial CM and IL-6 significantly increased the fraction of TUNEL-positive apoptotic cells in each differentiating culture (Control, 0.013+/−0.007; CM, 0.092+/−0.023; IL-6, 0.068+/−0.005, mean +/− s.e.m., n≧3). Although this increase was substantial, there was no increase in the relative apoptotic index within doublecortin positive vs. negative cells indicating that cell death was unlikely to select specifically against newborn neurons in vitro (FIG. 2F). The fraction of TUNEL-positive cells that co-labeled with doublecortin was 0.92+/−0.11 for controls (almost all TUNEL-positive profiles are also immunoreactive for Dcx), 0.89+/−0.07 of TUNEL profiles were Dcx positive in cultures treated with microglial CM and 0.83+/−0.02 (mean +/−sem) in cultures treated with IL-6 (see also supplemental FIG. 1).

Mitotic index (fraction of cells labeled with BrdU in 24 hours) in stem cell cultures was unaffected either by CM from stimulated microglia or by IL-6 (92 +/−2.8% in controls vs. 95+/−0.7% in CM or 95 +/−1.7% in IL-6 treated cultures). When the subset of spontaneously forming immature neurons was independently evaluated, there was a subtle but non-significant trend to reduced BrdU labeling within the neuronal progeny (88 +/−7.6% in controls vs. 82 +/−1.6% in IL-6 treated cultures). Thus, the effect of IL-6 on in vitro neurogenesis appears to induce both a non-specific decrease in cell survival as well as decreased accumulation of neurons, most likelydue to reduced neuronal differentiation rather than selective changes in the proliferation or death of neuroblasts or immature neurons. These findings, taken together with the effect of IL-6 over-expression in transgenic mice, implicate IL-6 as a regulator of hippocampal neurogenesis in neuroinflammation.

Signaling via gp130, the co-receptor of the IL-6 receptor, stimulates the Notch1 pathway, resulting in an increase in expression of the mammalian homolog of hairy-enhancer-of-split, Hes 1, transcription factor and antagonism of pro-neural basic helix-loop-helix (bHLH) genes and hippocampal neurogenesis during development. To determine if IL-6 treatment of adult stem cells leads to an increase in Hes 1 consistent with the reduction in neuronal cell fate, we performed “real-time” quantitative RT-PCR on total RNA extracted from neural precursors exposed for 60 hours to activated microglial CM or IL-6. Both CM and IL-6 caused a dramatic increase in Hes 1 mRNA expression (3.2- and 7.7-fold increase respectively, relative to control).

Having demonstrated that neuroinflammation alone is sufficient to inhibit neurogenesis, we then used the irradiation model to determine the relative role of inflammation in this irradiation-induced deficit. Adult rats were treated with indomethacin beginning 2 days prior to 10 Gy cranial X-irradiation and continuing daily for 2 months thereafter. Because rats are more radioresistent than humans, 10 Gy approximates a clinically relevant dose and is below the threshold to cause demyelination or overt vasculopathy in rats. This dose of X-irradiation was previously shown to spare roughly 30% of the NPC proliferative activity but completely ablate the production of neurons. X-irradiation was limited to a 1.5 cm cylinder centered over the cranium (remaining body parts were shielded). One month later, BrdU was administered systemically and at 2 months post-irradiation, brain tissues were analyzed for hippocampal neurogenesis.

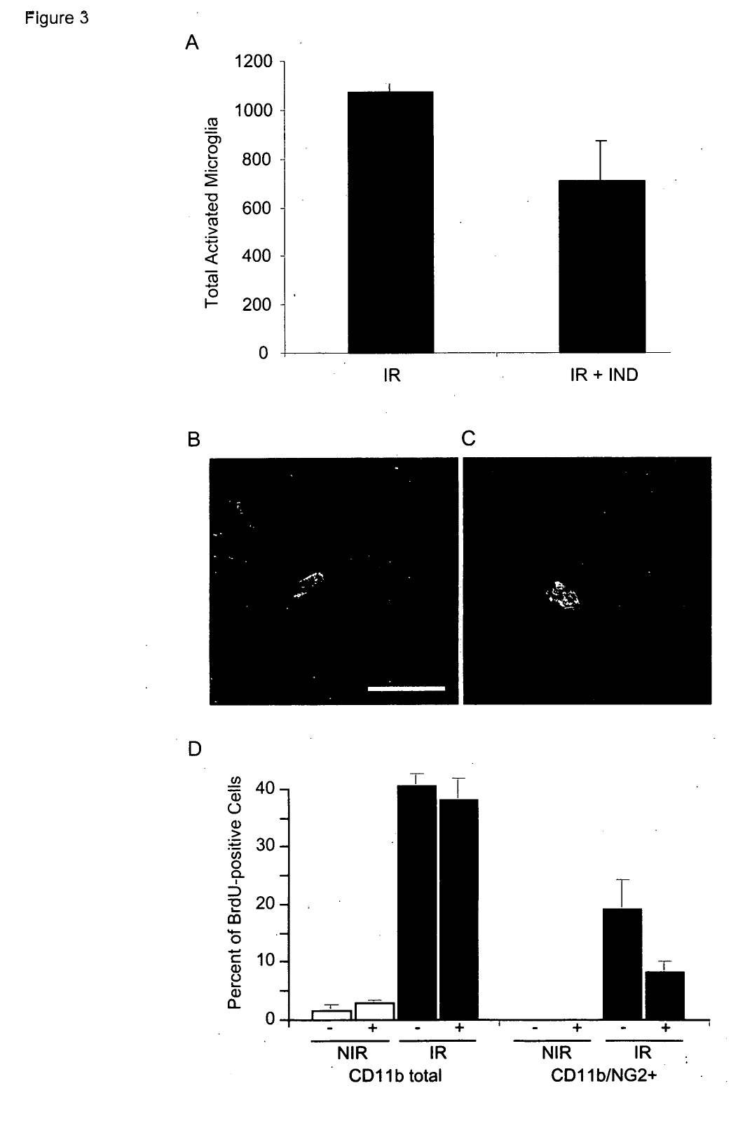

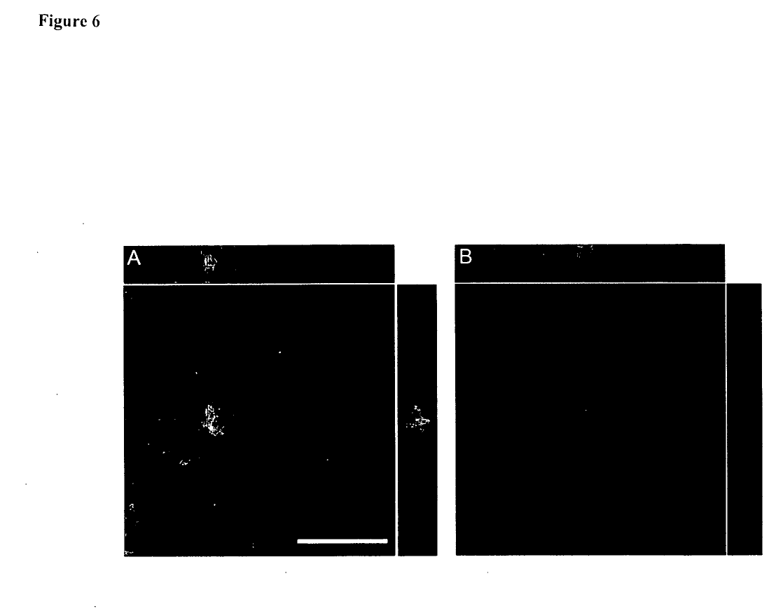

Irradiation causes a striking inflammatory response characterized by the persistence of activated microglia (FIG. 3A-C) relative to the minimal levels in normal control animals. Unbiased stereologic quantification of CD68 (ED1)-positive activated microglia in irradiated animals revealed that indomethacin treatment caused a 35% decrease in activated microglia per dentate gyrus (FIG. 3A). Many of these microglia were proliferative and a large fraction of all dividing cells within the dentate gyrus were labeled for the monocyte/microglia marker CD11b, which labels both activated and resting microglia (FIG. 3D). A subpopulation of CD11b+ microglia co-expressed the marker NG2 (FIG. 3C, D and FIG. 6), which represents peripheral blood monocytes/microglia that contribute to chronic neuroinflammatory lesions within the brain. Indomethacin was particularly effective at decreasing this CD11b/NG2+ subpopulation of infiltrating, proliferating peripheral monocytes following irradiation (FIG. 3D), suggesting an indomethacin-induced change in chemokine and/or integrin signaling that recruits trans-endothelial migration of immune cells following injury.

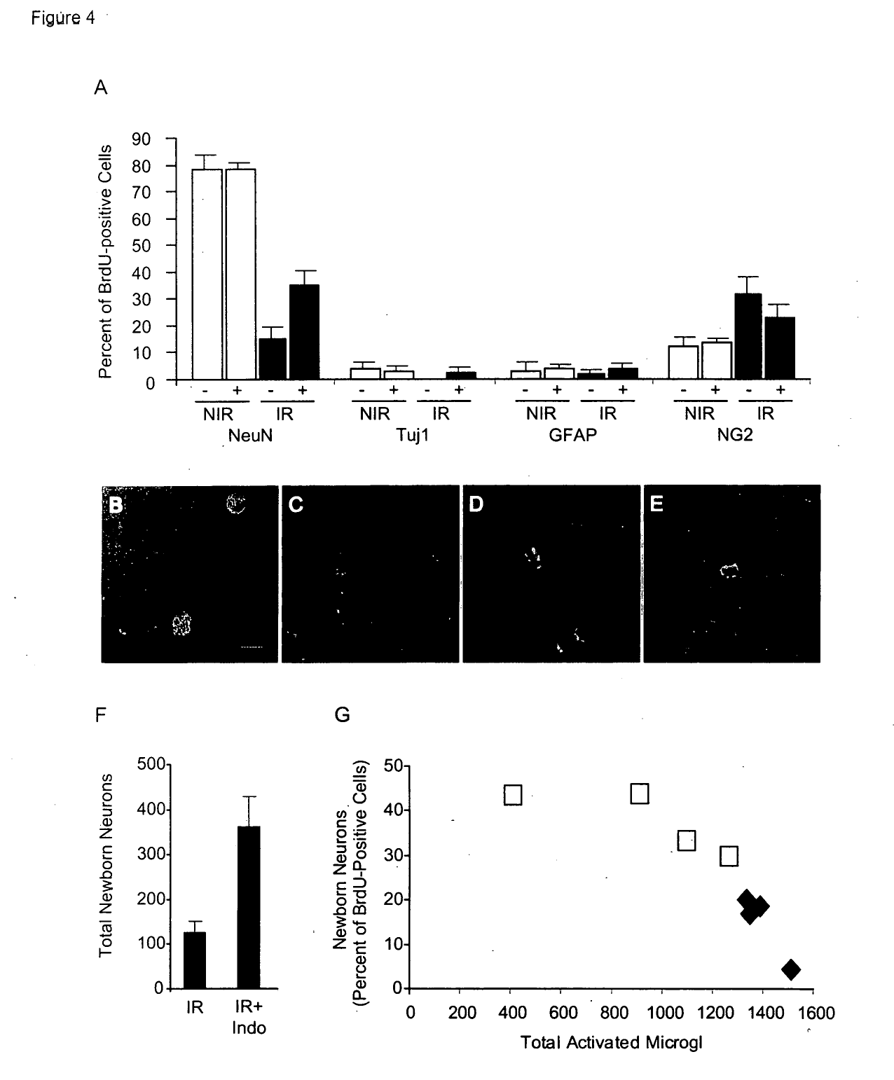

If inflammation were the primary cause of the lack of neurogenic signaling within the dentate subgranule zone, then inflammatory blockade would be expected to restore neurogenesis. Confocal microscopy was used to analyze the proportion of proliferative (BrdU+) cells that co-express markers (FIG. 4A) for mature neurons (NeuN) (FIG. 4B), immature neurons (type IlIl beta tubulin) (FIG. 4C), astrocytes (GFAP) (FIG. 4D) and immature oligodendrocytes (NG2+/CD11b−) (FIG. 4E). Indomethacin treatment in non-irradiated rats had no effect on cell fate relative to untreated, non-irradiated controls. Irradiation decreased the proportion of proliferative cells adopting a neuronal fate (FIG. 4A). Indomethacin treatment during and after irradiation exposure partially restored the relative proportion of proliferative cells adopting a neuronal fate relative to untreated, irradiated animals (37% vs. 15%, respectively; FIG. 4A-C).

Unbiased stereological quantification of total BrdU+ proliferative cells per neurogenic region (granule cell layer+subgranule zone) of the dentate gyrus revealed no significant difference in overall proliferation between indomethacin-treated and untreated irradiated animals (958±136 proliferative cells vs. 828±135 proliferative cells, respectively; control animals exhibited 1938±429 proliferative cells per neurogenic region). Correcting the fraction of proliferative cells adopting a neuronal fate for the total number of proliferative cells yields a significant increase in total newborn hippocampal neurons in indomethacin-treated, irradiated animals compared with untreated, irradiated animals (360±68 newborn neurons vs. 125±25 newborn neurons, respectively; FIG. 4F). This is a substantial increase in neurogenesis but still only 20% to 25% of the neurogenesis observed in naive animals (˜1600 newborn neurons).

To describe further the relationship between microglial inflammation and neurogenesis, we plotted neurogenesis against activated microglial load for each irradiated animal (FIG. 4G). Neurogenesis and inflammation show a striking negative correlation (R=−0.93 for activated microglial loads above 1000 per dentate gyrus; activated microglial load was ˜500 in controls.) suggesting that the extent of inflammation has a direct titrating role on neurogenesis within the adult dentate gyrus.

The present data indicate that inflammation itself can suppress neurogenesis and that chronic inflammation following radiation treatment contributes to the neural stem cell dysfunction that is linked to a progressive decline in learning and memory. Chronic microglial activation and peripheral monocyte recruitment with the accompanying increase in local pro-inflammatory cytokine production, including IL-6, emerge as potent anti-neurogenic components of brain injury. Both IL-6 and the IL-6 receptor/gp130 complex are expressed in the postnatal hippocampus, and hippocampal expression of the IL-6 receptor increases following systemic challenge with LPS. The IL-6 family of cytokines, including ciliary neurotrophic factor (CNTF) and leukemia inhibitory factor (LIF), belong to a category of signaling molecules termed “neurokines”. IL-6, like CNTF, promotes both astrogliogenesis and oligodendrogliogenesis, and it may be that IL-6 plays a role in inflammatory inhibition of neurogenesis by diverting stem cells into a glial program at the expense of neurogenesis. We find that gliogenesis is relatively well preserved in the irradiated microenvironment (FIG. 4A) and the in vitro data suggests that IL-6 inhibition of neurogenesis is primarily due to a blockade in neuronal differentiation ratherthan selective influences on cell death or proliferative activity.

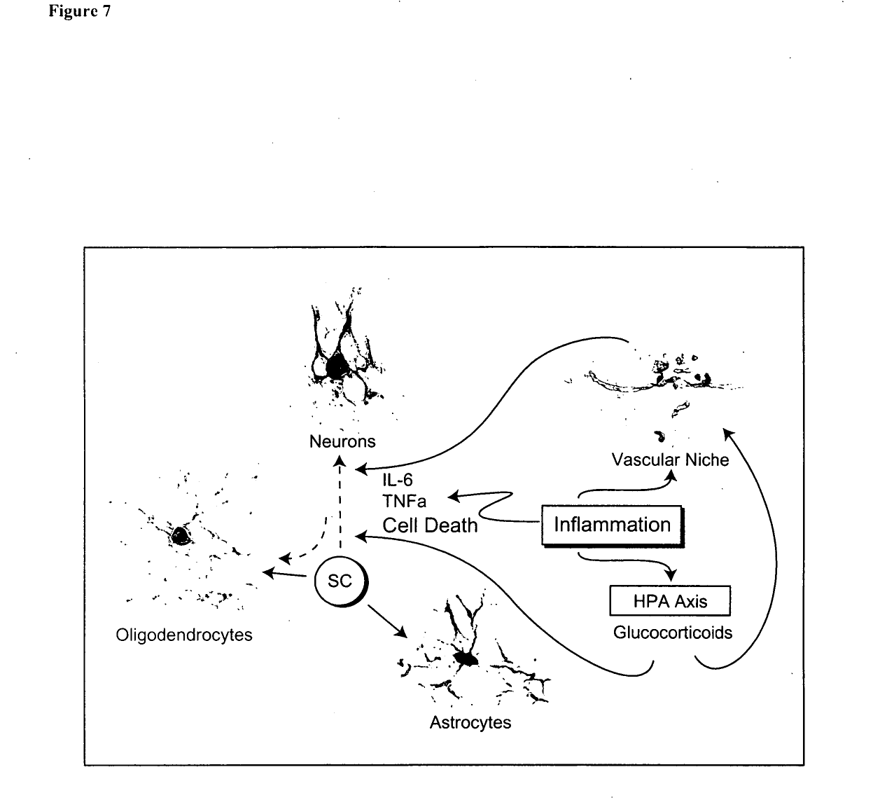

Inflammatory blockade with indomethacin decreased microglial activation and this may account for part of the restorative effect of indomethacin treatment on neurogenesis following irradiation. However, inflammatory blockade is accompanied by a broad spectrum of effects that could influence neurogeneis in several ways (FIG. 7). Restoration of neurogenesis with inflammatory blockade may involve a reduction in newborn cell death, and/or by attenuation of HPA axis activation. The subsequent decrease in pro-inflammatory cytokines and cognate decrease in serum glucocorticoid levels may contribute to restored neurogenesis. In addition, the microvasculature of the hippocampus is strongly implicated as a critical element of the neurogenic microenvironment and both endotoxin and irradiation-induced inflammation disrupts the association of proliferating progenitor cells with micro-vessels. The recruitment of circulating inflammatory cells is highly dependent on the endothelial status and elaboration of chemokines. One of the most robust effects of indomethacin in the present paradigm is the reduction in peripheral monocyte recruitment suggesting that the inflammatory status of endothelial cells (e.g., expression of chemokines and/or ICAM) may be normalized by indomethacin. Indeed, one known attribute of indomethacin treatment is the normalization of vascular permeability, which likely affects the neurogenic vascular microenvironment. Although IL-6 plays an important role, a narrow focus on IL-6 alone ignores the complexity of signaling that is altered in neuroinflammation and future experiments should address the more complex interactions of HPA axis, invading inflammatory cells, and alterations in the vascular niche of the hippocampal dentate gyrus. Neuroinflammation and microglial pathology are associated with many diseases of cognition in which memory loss features prominently, such as Alzheimer's Disease, Lewy Body Dementia, and AIDS Dementia Complex. Further, serum IL-6 levels in humans correlate with poor cognitive performance and predict risk of dementia. Clinical treatment with indomethacin and other NSAIDs ameliorates the risk and/or progression of memory loss. Our findings may shed some light on the potential contribution of inflammation-induced neurogenic blockade to memory pathology and on the mechanism of the beneficial effects of NSAID treatment in certain dementias.

Microglial cytokines also increase cell death and one action of inflammatory blockade may simply be rescue of newborn neurons that have been endangered by the inflammatory environment. Neurogenesis induced by hippocampal seizure activity is accompanied by a striking increase in the apoptosis of newborn neurons. Inflammation accompanies the seizure activity and cell death can be attenuated, in part, by treatment with an alternative anti-inflammatory drug, minocycline.

Decreasing microglial activation accounts for at least part of the restorative effect of indomethacin treatment on neurogenesis following irradiation. However, inflammatory blockade is accompanied by a broad spectrum of effects that could influence neurogeneis in several ways. Restoration of neurogenesis with inflammatory blockade may involve attenuation of HPA axis activation. The subsequent decrease in pro-inflammatory cytokines and cognate decrease in serum glucocorticoids may contribute to restored neurogenesis. In addition, the microvasculature of the hippocampus is strongly implicated as a critical element of the neurogenic microenvironment and both endotoxin and irradiation-induced inflammation disrupts the association of proliferating progenitor cells with micro-vessels. The recruitment of circulating inflammatory cells is highly dependent on the endothelial status and elaboration of chemokines. One of the most robust effects of indomethacin in the present paradigm is the reduction in peripheral monocyte recruitment suggesting that endothelial cell expression of chemokines and/or ICAM elaboration may be normalized by indomethacin. Indeed, one known attribute of indomethacin treatment is the normalization of vascular permeability, which likely has an impact on the neurogenic microenvironment.

Methods

Cell Culture:

Progenitor Cell Culture Adult hippocampus-derived neural precursor cells were isolated from adult rat hippocampus and cultured as previously described. Briefly, adult female Fisher-344 rats were deeply anesthetized with sodium pentobarbital and were dissected immediately. Hippocampi were enzymatically dissociated with papain (2.5 U/ml; Worthington, Freehold, N.J.)-dispase II (1U/ml; Boehringer Mannheim, Indianapolis, Ind.)-DNase I (250U/ml, Worthington) solution. Digested tissue was then washed with DMEM-10% fetal calf serum (FCS) and subsequently mixed with PBS-equilibrated Percoll solution to a final concentration of 35% Percoll. The Percoll solution was made by mixing nine parts of Percoll (Amersham Pharmacia Biotech, Uppsala, Sweden) with one part of 10×PBS. The cell suspension was then fractionated by centrifugation for 10 min at 1000×gravity. Floating myelin and tissue debris were discarded and the cell pellet re-suspended in 65% Percoll solution and fractionated again by centrifugation for 10 min at 1000×g. The floating neural precursors were collected, washed free of Percoll, and plated onto poly-L-ornithine/laminin-coated dishes in DMEM/F12 (1:1) containing 10% FCS medium for 24 hrs; then medium was replaced with serum-free growth medium consisting of DMEM/F12 (1:1) supplemented with N2 supplement (Life Technologies, Gaithersburg, Md.) and 20 ng/ml of human recombinant FGF-2 (Peprotech, Rocky Hill, N.J.). Cell lines were labeled via infection with replication deficient GFP-expressing recombinant retrovirus LZRS-CAMut4GFP. GFP-labeled cells were propagated in DMEM/F12 with 20 ng/ml bFGF, penicillin/ streptomycin/amphotericin B (Life Technologies), and N2 supplement (Life Technologies). Plastic tissue culture dishes were coated with 10 mg/ml polyornithine in dH20 overnight under UV illumination, rinsed 2× with dH20, recoated with 5 mg/ml mouse laminin (Life Technologies), incubated overnight at 37° C., and frozen for long-term storage at −80° C. Cells were fed every 2-3 days by 75% solution exchange and split 1:4 every 6-7 days after brief trypsinization and centrifugation. Freezing was in DMEM/F12/10% DMSO/BIT supplement (Stem Cell Technologies), and thawing from storage was in DMEM/F12/ BIT.

Microglia Culture BV-2 microglial cells were plated on uncoated plastic tissue culture plate and grown in DMEM:F12 (1:1) media with BIT supplement (Stem Cell Technologies).

Co-culture and production of conditioned media BV-2 murine microglia were stimulated with LPS (1μg/ml, Sigma, St Louis Mo.) for 12 hours. Control cultures were mock-stimulated with an equal volume of PBS. Cultures were then treated with trypsin, extensively washed and then re-plated with an equal number of GFP-positive hippocampal stem/progenitor cells on laminin-coated dishes (no LPS was present in the co-culture). Co-culture was done in differentiation media, DMEM:F12 (1:1) with BIT supplement, 1% fetal bovine serum, 100 nM all trans-retinoic acid, 2 ng/ml FGF-2 and 1 Ong/ml NT3 for 60 hours. To prepare conditioned media from stimulated and non-stimulated microglia, microglia were treated with LPS or PBS (unstimulated controls) for 24 hours and then washed to remove LPS from stimulated cultures. Fresh differentiation medium was then incubated with the microglia overnight and then removed, sterile filtered and diluted with fresh differentiation media (1:1) prior to adding to neural stem/progenitor cell cultures. In a modified version of this experiment, a neutralizing anti-IL-6 antibody (1ug/ml final concentration in culture medium, R&D systems, Minneapolis, Minn.) was added to the conditioned media from microglia. After 60 hrs, co-cultured or conditioned-media-treated stem/progenitor cells were fixed with 4% buffered paraformaldehyde and immunostained for doublecortin using goat anti-doublecortin (Dcx) (1:500, Santa Cruz Biotechnology, Santa Cruz, Calif.). DAPI was used to identify cell nuclei. Fluorescent photomicrographs were taken at systematically sampled positions within each well and changes in doublecortin expression relative to control cultures scored by unbiased quantification of the average signal intensity in positive cells (i.e., fluorescence intensity above a background threshold of Dcx fluorescence measured in undifferentiated cells using Photoshop).

Recombinant cvtokines Recombinant rat cytokines (R&D Systems, Minneapolis, Minn.), interleukin 1β (6-50 ng/ml), tumor necrosis factor α (2-20 ng/ml), interferon γ (1-5ng/ml) and interleukin-6 (6-50 ng/ml) were added to hippocampal precursor cells cultured on laminin-coated plates in differentiation media, DMEM:F12 (1:1) with BIT supplement, 1% fetal bovine serum, 100 nM all trans-retinoic acid, 2 ng/ml FGF-2 and 1 Ong/ml NT3 for 60 hours. Cells were immunostained for doublecortin, type III β-tubulin, GFAP, or NG2 and analyzed as above.

Proliferation and survival assays BrdU was added to the culture media from hour 24 to hour 48 of the 60 hour paradigm. Immunocytochemistry and confocal microscopy was then used to determine the fraction of GFP+ cells that labeled with BrdU, TUNEL, or type III β-tubulin or doublecortin. TUNEL staining was performed with Apoptag Red (Serologicals, Norcross, Ga.). The fraction of total (DAPI+) nuclei that were TUNEL+ were determined, as well as the fraction of doublecortin positive cells that were also TUNEL+.

Total RNA isolation, cDNA synthesis, and SYBR Green real-time quantitative RT-PCR. Total RNA was isolated from neural precursor cell cultures using RNeasy mini kit (Qiagen) and synthesis of cDNA was performed using the SuperScript First-strand Synthesis System for RT-PCR (Invitrogen). Quantitative SYBR Green real time PCR was carried out as described previously. Briefly, each 25 μl SYBR green reaction consisted of 5 μl of cDNA (50 ng/pl), 12.5 μl of 2× Universal SYBR Green PCR Master Mix (PerkinElmer Life Sciences) and 3.75 μl of 50 nM forward and reverse primers. Optimization was performed for each gene-specific primer prior to the experiment to confirm that 50 nM primer concentrations did not produce nonspecific primer-dimer amplification signal in no-template control tubes. Primer sequences were designed using Primer Express Software. Quantitative RT-PCR was performed on ABI 5700 PCR instrument (PerkinElmer Life Sciences) by using 3-stage program parameters provided by the manufacturer as follows; 2 min at 50° C., 10 min at 95° C., and then 40 cycles of 15 s at 95° C. and 1 min at 60° C. Specificity of the produced amplification product was confirmed by examination of dissociation reaction plots. A distinct single peak indicated that single DNA sequence was amplified during PCR. In addition, end reaction products were visualized on ethidium bromide-stained 1.4% agarose gels. Appearance of a single band of the correct molecular size confirmed specificity of the PCR. Each sample was tested in five copies with quantitative PCR, and samples obtained from three independent experiments were used to calculate the means and standard deviations. Primers were as follows (F=forward, R=reverse):

| | |
|---|---|
| GAPDH F | AAGAGAGAGGCCCTCAGTTGCT |
| GAPDH R | TTGTGAGGGAGATGCTCAGTGT |
| MASH1 F | GACAGGCCCTACTGGGAATG |
| MASH1 R | CGTTGTCAAGAAACACTGAAGACA |
| HES1 F | CGGCTTCAGCGAGTGCAT |
| HES1 R | CGGTGTTAACGCCCTCACA |
| HES5 F | GGAGGCGGTGCAGTTCCT |
| HES5 R | GGAGTGGTAAAGCAGCTTCATC |
| NEUROD F | GGACAGACGAGTGCCTCAGTTC |
| NEUROD R | TCATGGCTTCAAGCTCATCCTCCT |

Indomethacin administration. We chose the non-streroidal anti-inflammatory drug indomethacin as an anti-inflammatory agent for its potency, ability to penetrate the blood-brain-barrier (BBB), demonstrated efficacy in decreasing microglial inflammation in vitro and in vivo, and particular ability to decrease monocyte/microgilial migration and elaboration of pro-inflammatory cytokines. Indomethacin inhibits cyclo-oxygenase (COX; type 1>2), thereby decreasing production of the prostaglandin arachadonic acid metabolites that broadly contribute yo microglial recruitment and activation. Additionally, indomethacin agonizes the transcription factor peroxisome proiferator-activator-γ.(PPAR-γ)that inhibits the elaboration of pro-inflammatory cytokines in monocytes/microglia.

Adult female Fisher 344 rats (160-180 grams) were given indomethacin (Sigma, St Louis, Mo.) 2.5 mg/Kg, administered either i.p. (in 5% bicarbonate) or mixed into soft dog food (Pedigree, Kal Kan foods, Inc, Vernon, Calif.) every 12 hours for either 1 week (i.p administration paradigm) or two months (dog food paradigm), beginning the day before LPS exposure or irradiation and ending on the day of sacrifice. Control animals were given either vehicle (5% bicarbonate) injections i.p. or plain dog food. To ensure complete consumption of food and medication in the 2 month paradigm, rats were restricted to 80% of their ad lib intake. Rats continued to gain weight and exhibited normal grooming behavior throughout the two-month experiment. Serum indomethacin levels achieved with this paradigm ranged from 2.7 to 3.7 ug/ml (the human therapeutic index is 1.0 to 2.0 ug/ml).

LPS exposure. Bacterial lipopolysaccharide (LPS, Sigma, St Louis, Mo.) was administered in sterile saline by intraperitoneal injection at a dose of 1 mg/Kg one time. This caused mild sickness behavior (decreased grooming, decreased locomotor activity, increased piloerection) for approximately 2 days, resolving by the 3^{rd} day. The dose of LPS chosen causes mild sickness behavior in rats that resolves within 1-2 days; the dose of LPS used to induce endotoxic shock is 10-fold higher than that used in the present study.

Irradiation Adult female Fisher 344 rats were anesthetized with ketamine and xylazine and exposed to cranial irradiation using a Philips orthovoltage X-ray system operated at 200 kVp and 20 mA. A single dose of 10 Gy was delivered to the cranium of each rat with shielding of the body, neck, eyes and snout. Dosimetry was done using TLD dosimeters (K & S Associates, Nashville, Tenn.) buried in the hippocampi, cerebellum, mouth, eyes and ears of euthanized rats. The corrected dose rate was approximately 44.5 cGy/minute. Sham-irradiated controls for all experiments received anesthesia only.

BrdU iniections and tissue preparation. Animals were injected intraperitoneally with BrdU once each day for 6 days. Animals were then anesthetized and sacrificed on the 28^{th} day after the initial BrdU injection by transcardial perfusion with 4% paraformaldehyde. Brains were removed and postfixed overnight and then equilibrated in phosphate buffered 30% sucrose. Free floating 40 μm sections were collected on a freezing microtome and stored in cryoprotectant as previously described.

Immunohistochemistry and immunofluorescent staining. Free floating sections were immunostained as previously described using the following primary antibodies and working concentrations: mouse anti-NeuN (1:4, gift from R. Mullen); guinea pig-anti GFAP (1:800, Harlan, Indianapolis, Ind.); mouse anti-type III ptubulin (Tuj-1, 1:500, BerkeleyAntibody Co., Richmond, Calif.); rabbit anti-NG2 (1:200, Chemicon, Temecula, Calif.); mouse anti-rat CD11b (1:200, Serotec, Oxford, U.K.); mouse anti-ED-1 (1:100, Research Diagnostics Inc., Flanders, N.J.); biotinylated-Lycopersicon esculentum (tomato) lectin (1:200, Vector, Burlingame, Calif.).

Confocal microscopy. All confocal microscopy was performed using a Zeiss 510 confocal microscope. Appropriate gain and black level settings were determined on control tissues stained with secondary antibodies alone. Upper and lower thresholds were always set using the range indicator function to minimize data loss through saturation.

Cell counting and unbiased stereology. All counts were limited to the hippocampal granule cell layer proper and a 50 μm border along the hilar margin that included the neurogenic subgranule zone. The proportion of BrdU cells displaying a lineage-specific phenotype was determined by scoring the co-localization of cell phenotype markers with BrdU using confocal microscopy. Split panel and z-axis analysis were used for all counting. All counts were performed using multi-channel configuration with a 40× objective and electronic zoom of 2. When possible, 100 or more BrdU-positive cells were scored for each marker per animal. Each cell was manually examined in its full “z”-dimension and only those cells for which the nucleus was unambiguously associated with the lineage-specific marker were scored as positive. The total number of BrdU-labeled cells per hippocampal granule cell layer and subgranule zone was determined using diaminobenzadine stained tissues. In a separate series, the total number of ED1-labeled cells per dentate gyrus was also determined using diaminobenzadine stained tissue. Stained BrdU-positive nuclei or ED1-positive cells were scored under light microscopy using Microbrightfield Stereo Investigator software and the Fractionator method. Overestimation was corrected using the Abercrombie method for nuclei with empirically determined average diameter of 13 μm within a 40 μm section.

### Example 2

### Behavioral Aspects of Inflammation-induced Deficits

LPS-induced inflammatory response impairs performance in the Bames maze. It has been reported previously that irradiation and LPS-induced inflammation impair performance in hippocampus-dependent spatial tasks. The present studies confirm that LPS treatment impaired recall of a goal position learned prior to LPS treatment. The Barnes maze is a 6-foot diameter bright white platform with 8 escape boxes under holes in the rim of the platform. All escape holes are blocked except one and the rat is initially placed in the goal box for familiarization. The rat is then placed in the center of the maze and is allowed to find its way back to the goal box to escape the aversive brightly lit platform. Learning is seen as a decrease in the distance traveled to reach the goal box with consecutive trials on a given day and in repeat sessions on sequential days. One measure of recall is to determine distance traveled when the animal is tested on the same task after a time delay or after experimental treatments.

To determine if there were differences in recall following LPS treatment, animals were trained on the Barnes maze for 5 consecutive days prior to LPS treatment. LPS was given on day 0 and animals tested for recall on day 7. Low-dose intraperitoneal LPS treatment makes animals feel mildly ill and they will reduce their water and food intake for a period of one or two days. There is a temporary weight loss that is fully recovered by day 7. Although all animals showed normal weights and were indistinguishable on independent measures of locomotor activity, there was a significant increase in distance traveled in LPS vs. vehicle groups indicating a measurable LPS effect in this paradigm. This deficit disappeared by two weeks but re-training animals to a new goal box position showed that LPS treatment two weeks prior still impaired the acquisition of a new task. The effects of indomethacin were tested on both acquisition and retention following LPS treatment or irradiation.

To determine if treatment with indomethacin influenced the LPS effects on learning and memory, animals were trained on the Barnes maze for 5 days prior to treatment with a single intraperitoneal injection of LPS. Animals were simultaneously treated with indomethacin twice daily (2.5 mg/kg), either in edible treats or by intraperitoneal injection in aqueous vehicle. Animals were weighed daily and after 7 days tested for memory retention on the Barnes maze. On days 8-11, animals were re-trained for their ability to learn the position of a new goal box in the Barnes maze. Animals were then tested on day 14 for the ability to remember the position of the second goal box. LPS caused a ˜14% increase in the distance traveled to the goal box learned prior to LPS treatment, indicating impaired spatial memory. LPS caused a significant increase (˜28%) in the distance traveled during day 8 trials used to learn the position of the new goal box (indicating impaired spatial learning) but all animals eventually learned the position of the goal box by day 11 (no difference in distance traveled between LPS and control groups).

Indomethacin treatment by intraperitoneal injection alone (in the absence of LPS) caused animals to perform more poorly in all tasks at all time points indicating a drug-induced impairment in both learning and memory (when administered by IP injection). In contrast, oral indomethacin treatment alone had no measurable effects on learning and memory at any timepoint indicating that oral administration was well tolerated.

Oral administration of indomethacin was able to completely reverse the effects of LPS for both memory retention and in learning the position of the second goal box. This indicates that indomethacin is able to block the effects of LPS that negatively affect learning and memory. This also indicates that intraperitoneal administration of indomethacin (while able to restore neurogenesis as measured in our prior work) is itself not well tolerated and negatively influences rat performance in learning and memory tasks. This is an important observation suggesting that IP administration of drugs may cause sufficient stress to mask the behavioral effects being studied.

Morris Water Maze. The Morris water maze is a large 6′ diameter pool of water in which a submerged 4″ diameter platform is hidden just below the surface of the water. Rats placed into the pool will swim in an attempt to escape and will find the platform accidentally or, after 90 seconds of swimming will be placed on the platform and thereby learn the platform position. Improving recall of the platform position is represented in a shortening of the path the animal takes to reach the platform.

Prior to treatment, adult female rats are given a water maze baseline trial in which they are placed in the pool with no platform for 90 s to habituate to the environment and to measure individual variability in swimming ability (swim speed and swim distance) as well as quadrant biases. Animals are subsequently trained with an additional 4 trials/day over 4 days in which they are given 90 s to locate a platform hidden beneath the water. If unable to locate the platform, the animals are manually placed on the platform and allowed to sit for 20 seconds. Latency, path length and heading angle are the variables that are recorded using an HVS tracking system to establish a baseline measure how well each rat learns the location of the hidden platform as combined, these variables are considered to be valid and reliable measures of hippocampus-dependent learning in water maze tasks. In addition, “search error” (average distance from the platform during the trial) will be analyzed as it is a more sensitive measure of spatial learning than latency, swim path length and heading angle for spatial learning deficits in aged rats. On the 5th day of pre-training, the animals participate in a probe trial in which they are placed in the pool with no platform for 90 seconds to measure the strength of their spatial learning or retention of information about the location of the hidden platform. The fraction of time and fraction of swim path length spent in the platform quadrant indicates better retention of this information.

Animals are then entered in the radiation or LPS paradigms. At the end of the treatment strategies (2 weeks after LPS or 8 weeks after irradiation), the animals are again tested for spatial memory. First, animals are given a probe trial with no platform to measure quadrant swim speed, path length, and perseveration in the pre-test platform quadrant. Then, the animals are tested in using a reversal paradigm where the platform is placed in the opposite quadrant and animals trained as in the pre-test. Difficulty in acquiring the new platform position indicates either impaired acquisition or abnormal preservation (i.e., continued preference to search in the old platform quadrant even when the new platform position is presented to the animals. Differences in acquisition between pre-and post-experiment performances are evaluated for each animal and these differences contrasted between groups. As in the Barnes maze, a group size of 12 is sufficient to detect small changes in retention or acquisition.

The primary goal in these studies is to correlate neurogenesis to either acquisition or retention of a spatial memory. The Barnes maze testing provides a user-friendly paradigm (rats stay dry) and will be our primary analysis tool. The Morris water maze testing is done on subsets of animal groups to validate the Barnes maze data using a separate paradigm. Animals are scored for latency (total time to acquire the hidden platform or goal box), speed, path angle relative to the platform or goal box, and total path distance. In the probe trials (platform removed or goal box hidden), the total time and path distance within each quadrant will be scored. The four trials per day are binned into a daily block (average of 4 trials) and dependent variables (latency, path-length and average path-speed to reach the goal) will be analyzed using repeated-measures ANOVAs with the following conditions: 1. non-irradiated vs. irradiated; 2. irradiated vs. irradiated/indomethacin; 3. non-irradiated vs. irradiated/indomethacin. Identical analysis will be used in LPS paradigms. Similar analysis will be prepared for individual animals to determine the significance of changes observed between training session (days 1, 2, 3, 4) or between pre-experiment and post-experiment data for a given animal. A Newman-Keul strategy will be used for post hoc analysis.

These data demonstrate the effectiveness of anti-inflammatory agents in preventing cognitive defects associated with neuroinflammation. The effectiveness of the methods on the treatment of human subjects is similarly evaluated, through learning and memory tasks, and may be further evaluated using functional criteria known in the art, for example through the use of fMRI.

### Example 3

### The Chemokine Monocyte Chemoattractant Protein-1 (MCP-1) is Necessary for Irradiation-induced Inhibition of Neurogenesis

Neuroinflammation inhibits adult hippocampal neurogenesis through both a specific block in neuronal differentiation and a generalized decrease in newborn cell survival. As shown above, anti-inflammatory therapy with the NSAID indomethacin restores neurogenesis following cranial radiation exposure. Because one of the most robust effects of indomethacin treatment was to decrease the number of infiltrating blood monocytes, the population of recruited monocytes/macrophage may be an important component of the neuroinflammatory response to irradiation, and recruited monocytes/macrophages may play a pivotal role in inflammatory inhibition of neurogenesis.

Monocyte chemoattractant protein-1 (MCP-1) is a CC family chemokine (i.e., chemoattractant cytokine), Rollins (1997) Blood 90, 909-928, that is produced by astrocytes and microglia in response to injury or inflammatory cytokines. MCP-1 is necessary for monocyte recruitment to sites of inflammation. The MCP-1 receptor, CCR2, is expressed by many cell types in the brain including monocytes, neural progenitor cells, smooth muscle and endothelial cells (see Banisadr et al. (2002) J. Neurochem. 81, 257-269).

Loss of MCP-1 function in mice may lead to a less severe defect in neurogenesis following irradiation. This may occur by reducing peripheral monocyte recruitment and/or by reducing the inflammation-induced changes to the stem cell's vascular microenvironment or via MCP-1 action on the precursor itself. The following data address this question, and demonstrate the important role of MCP-1 in mediating the adverse effects of neuroinflammation.

Methods

Adult mice were treated with 10 Gyx-irradiation limited toa 1 cm column centered over the cranium and allowed to recover for one month. The MCP −/− mice are as described by Lu et al. (1998) J. Exp. Med. 187:601-608; and the control wild-type mice are otherwise genetically matched to the knockout mice. 50 mg/kg BrdU was then administered dailyfor6 days and animals allowed to survive for an additional 3 weeks. Brains were evaluated for neurogenesis 2 months after irradiation (one month after the initial BrdU injection). Neurogenesis was measured as surviving, BrdU-positive neurons in the dentate gyrus of the hippocampus.

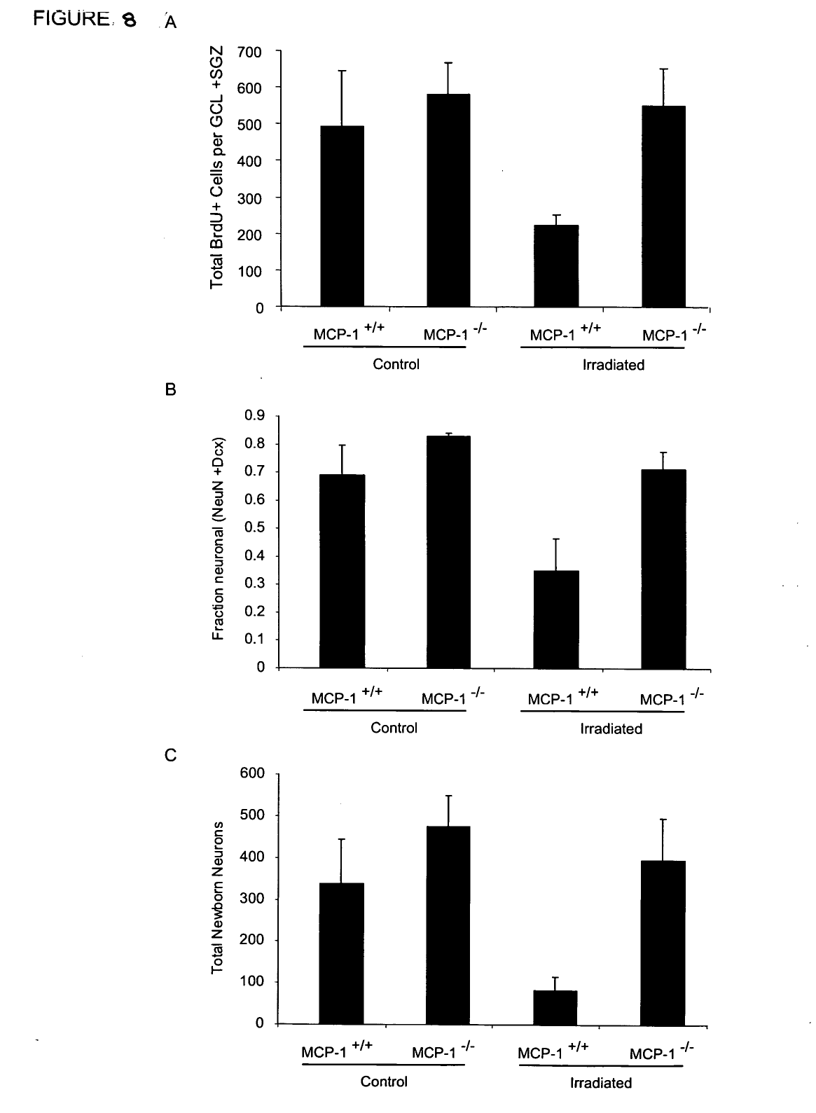

Proliferative cell fate was determined using immunofluorescent staining and confocal microscopy. The percent of BrdU-iabeled cells that adopt a neuronal cell fate (NeuN plus Doublecortin) is shown in FIG. 8B. The total number of newborn neurons was also estimated by correcting the proportion of BrdU-labeled neurons for total number of BrdU-positive cells per hippocampal dentate gyrus (FIG. 8C).

Results

Both MCP-1 −/− and wild type animals had a similar density of microglia in the hippocampus, demonstrating that MCP-1 is not necessary for developmental colonization of brain with microglial cells.

However, in the MCP-1 −/− mice, the monocyte/microglia response after irradiation was attenuated relative to the wild type mice. Total microglial (Iba1+) cell densitywas reduced; and cell bodies were smaller, indicating decreased activation. Staining with a reagent specific for activated microglia (CD68⁺) showed a lowered density; and the staining intensity was reduced, suggesting impaired microglial activation in MCP-1 null animals.

In normal adult rats, irradiation causes a 70% decrease in the number of newbom cells that accumulate in the hippocampus, although the number of progenitor cells that can be isolated from the irradiated hippocampus is not significantly different from that of a non-irradiated animal. This indicates a defect in either proliferation and/or survival of progenitor progeny after irradiation. MCP-1 could contribute to this defect by altering the precursor cells' local microenvironment, i.e., either by altering vascular the vascular and astrocytic microenvironment of the precursor cells in the hippocampal subgranular zone and/or by subsequent extravasation of monocytes into this vascular niche.

To determine if absence of MCP-1 influenced the severity of irradiation-induced precursor dysfunction in mice, the accumulation of BrdU-positive cells was evaluated in MCP-1 null animals vs. wild type controls. Using unbiased stereological quantification of total BrdU+ cells in the neurogenic region of the hippocampus (granule cell layer plus subgranular zone) we observed an expected decrease in the total number of BrdU+ cells in irradiated wild type mice at one month after BrdU labeling, indicating similar radiation response between mice and rats (55% decrease relative to non-irradiated wild type controls, FIG. 8A, P<0.05, n=3. ) In stark contrast, MCP−/− mice exhibited normal levels of total BrdU+ cells following irradiation (FIG. 8A, n=3 per group.)

Wild type mice exhibited the expected decrease in the proportion of newborn cells that adopt a neuronal phenotype following irradiation relative to non-irradiated controls (FIG. 8B, P<0.05, n=3. ). When corrected for total number of BrdU cells, the net loss of neurogenesis in wild type animals was greater than 75% (FIG. 1C, P<0.05, n=3).

There was no difference in neurogenesis in non-irradiated MCP-1 −/− null vs. wild type mice, indicating that neurogenesis is normal in the absence of MCP-1 (FIG. 8B-C, P=0.26, n=3. ) Strikingly, between these two groups there was no detectable decrease in either the proportion of BrdU+ cells adopting a neuronal phenotype nor the total number of newborn neurons in MCP-1 −/− mice following irradiation. These data demonstrate that neurogenesis was completely unaffected following irradiation of MCP-1 null animals (FIG. 8A-C), and therefore that MCP-1 activity is necessary for the detrimental effects of irradiation on adult hippocampal neurogenesis.

The resistance of MCP-1 −/− mice to the effects of irradiation on neurogenesis at two months following irradiation is a surprisingly robust finding, and speaks to either the importance of MCP-1 in inflammatory cell recruitment to brain and status of the precursor cell's microenvironment or to a possible direct effect of MCP-1 on neural precursor cells. Given this very robust effect, antagonists or inhibitors of MCP-1, which may include small molecule inhibitors, siRNAs, biologic effector molecules, and other modulators of MCP-1 or MCP-1 receptor (CCR2) action will have profound restorative effects on neurogenesis following irradiation.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. All technical and scientific terms used herein have the same meaning as commonly understood to one of ordinary skill in the art to which this invention belongs unless clearly indicated otherwise.

What is claimed is:

1. A method of protecting an individual from a loss of neurogenesis capacity resulting from neuroinflammation, the method comprising:
contacting said individual with a dose of an anti-inflammatory agent effective to reduce neuroinflammatory activity by recruitment or activation of monocyte/microglial cells;
wherein said loss of neurogenesis capacity is reduced.

2. The method according to claim 1, wherein said inflammation and activation of microglial cells is the result of cranial irradiation.

3. The method according to claim 2, wherein said radiation is ionizing radiation.

4. The method according to claim 3, wherein said contacting is performed prior to said irradiation.

5. The method according to claim 3, wherein said contacting is performed subsequent to said irradiation.

6. The method according to claim 1, wherein said anti-inflammatory agent is a non-steroidal anti-inflammatory agent.

7. The method according to claim 1, wherein said anti-inflammatory agent specifically blocks IL-6 activity.

8. The method according to claim 1, wherein said anti-inflammatory agent blocks MCP-1 activity.

9. The method according to claim 1, wherein said loss of neurogenesis capacity is associated with Alzheimer's disease, Parkinson's disease, multiple sclerosis, depression, bipolar disorder or Cushing's disease.

10. The method according to claim 1, wherein said loss of neurogenesis capacity is associated with Lewy Body dementia, Frontotemporal dementia/Pick's disease, AIDS dementia complex, dementia puligistica and chronic cognitive dysfunction following head trauma, prion-associated dementia such as Creutzfeldt-Jacob disease, cognitive dysfunction following chronic seizure disorders or an episode of status epilepticus, or cognitive dysfunction following encephalitis or meningitis, amyotrophic lateral sclerosis (ALS)/parkinsonian/dementia complex of Guam.

11. The method according to claim 1, wherein said loss of neurogenesis capacity is associated with accute injury.

12. The method according to claim 1, wherein said loss of neurogenesis capacity is associated with pre- or peri-natal ischemia/hemorrhage associated with the developmental dysregulation of stem/progenitor cells in early life.

13. The method according to claim 1, wherein said loss of neurogenesis capacity results from the attention by inflammation of neurogenesis in response to surgical interventions, injury, or disease.

14. The method according to claim 1, wherein said neurogenesis is central nervous system neurogenesis.

15. The method according to claim 1, wherein said neurogenesis is peripheral nervous system neurogenesis.

16. The method according to claim 15, wherein said loss of neurogenesis capacity is associated nerve injury due to trauma, surgery, cancer, multiple sclerosis, ALS, or other motor neuron disease where endogenous or grafted progenitor/stem cells are influenced by immune mechanisms.

17. A method of protecting an individual from a loss of neurogenesis capacity resulting from neuroinflammation as a result of transplantation of neural progenitors to the central nervous system, the method comprising:
contacting said individual with a dose of an anti-inflammatory agent effective to reduce neuroinflammatory activity by recruitment or activation of monocyte/microglial cells;
wherein said loss of neurogenesis capacity is reduced.

18. A method of screening a candidate agent for modification or detection of stem or progenitor cell dysfunction, the method comprising:
determining the effect of said agent on stem or progenitor cell dysfunction in the absence and presence of an anti-inflammatory agent.

19. A method of screening a candidate agent for activity in protecting an individual from a loss of neurogenesis capacity resulting from neuroinflammation, the method comprising:
contacting a model for neuroinflammation with a candidate agent, and determining the effectiveness of said agent on neurogenesis.

20. A method of screening a candidate agent for activity in protecting an individual from a loss of neurogenesis capacity resulting from neuroinflammation, the method comprising:
contacting a model for neuroinflammation with a candidate agent, and determining the effectiveness of said agent on inhibiting expression of MCP-1.
